SEQUENCE LISTING (156 sequences)

SEQ ID NO: 1 - protein, 18 aa, Artificial Sequence
  synthetic polypeptides corresponding to amino acid 248 to 265 of ASB11
TDYGANLKRRNAQGKSAL

SEQ ID NO: 2 - DNA, 21 bp, Artificial Sequence
  synthetic polynucleotides ASB11 qPCR primer F
cctgctaaccgactatggagc

SEQ ID NO: 3 - DNA, 21 bp, Artificial Sequence
  synthetic polynucleotides ASB11 qPCR primer R
taggaggaatcgttcgagtgg

SEQ ID NO: 4 - DNA, 21 bp, Artificial Sequence
  synthetic polynucleotides ATF6 qPCR primer F
tcctcggtcagtggactctta

SEQ ID NO: 5 - DNA, 23 bp, Artificial Sequence
  synthetic polynucleotides ATF6 qPCR primer R
cttgggctgaattgaaggttttg

SEQ ID NO: 6 - DNA, 21 bp, Artificial Sequence
  synthetic polynucleotides BIK qPCR primer F
gacctggaccctatggaggac

SEQ ID NO: 7 - DNA, 21 bp, Artificial Sequence
  synthetic polynucleotides BIK qPCR primer R
cctcagtctggtcgtagatga

SEQ ID NO: 8 - DNA, 22 bp, Artificial Sequence
  synthetic polynucleotides GAPDH qPCR primer F
gtctcctctgacttcaacagcg

SEQ ID NO: 9 - DNA, 22 bp, Artificial Sequence
  synthetic polynucleotides GAPDH qPCR primer R
accaccctgttgctgtagccaa

SEQ ID NO: 10 - DNA, 23 bp, Artificial Sequence
  synthetic polynucleotides NFYB qPCR primer F
atgacaatggatggtgacagttc

SEQ ID NO: 11 - DNA, 21 bp, Artificial Sequence
  synthetic polynucleotides NFYB qPCR primer R
ctagccacgtttgctattgga

SEQ ID NO: 12 - DNA, 22 bp, Artificial Sequence
  synthetic polynucleotides NFYC qPCR primer F
ggaggatttggtggtactagca

SEQ ID NO: 13 - DNA, 21 bp, Artificial Sequence
  synthetic polynucleotides NFYC qPCR primer R
gcactcggaagtctttcactg

SEQ ID NO: 14 - DNA, 21 bp, Artificial Sequence
  synthetic polynucleotides p53 qPCR primer F
acttgtcgctcttgaagctac

SEQ ID NO: 15 - DNA, 22 bp, Artificial Sequence
  synthetic polynucleotides p53 qPCR primer R
gatgcggagaatctttggaaca

SEQ ID NO: 16 - DNA, 21 bp, Artificial Sequence
  synthetic polynucleotides PERK qPCR primer F
acgatgagacagagttgcgac

SEQ ID NO: 17 - DNA, 21 bp, Artificial Sequence
  synthetic polynucleotides PERK qPCR primer R
atccaaggcagcaattctccc

SEQ ID NO: 18 - DNA, 21 bp, Artificial Sequence
  synthetic polynucleotides XBP1 qPCR primer F
ccctccagaacatctccccat

SEQ ID NO: 19 - DNA, 21 bp, Artificial Sequence
  synthetic polynucleotides XBP1 qPCR primer R
acatgactgggtccaagttgt

SEQ ID NO: 20 - DNA, 20 bp, Artificial Sequence
  synthetic polynucleotides XBP1s qPCR primer F
aagcacctctcagcccctca

SEQ ID NO: 21 - DNA, 20 bp, Artificial Sequence
  synthetic polynucleotides XBP1s qPCR primer R
gctttgggcagtggctggat

SEQ ID NO: 22 - DNA, 21 bp, Artificial Sequence
  synthetic polynucleotides XBP1s (intron) PCR primer F
ttacgagagaaaactcatggc

SEQ ID NO: 23 - DNA, 22 bp, Artificial Sequence
  synthetic polynucleotides XBP1s (intron) PCR primer R
gggtccaagttgtccagaatgc

SEQ ID NO: 24 - DNA, 22 bp, Artificial Sequence
  synthetic polynucleotides A (-63 ˜ +165) ChIP-qPCR primer F
tctcggtaacaggagcactgag

SEQ ID NO: 25 - DNA, 24 bp, Artificial Sequence
  synthetic polynucleotides A (-63 ˜ +165) ChIP-qPCR primer R
gatctcttctgctatcctagccgc

SEQ ID NO: 26 - DNA, 21 bp, Artificial Sequence
  synthetic polynucleotides B (-304 ˜ -86) ChIP-qPCR primer F
ctgtggaagccaagaagtttc

SEQ ID NO: 27 - DNA, 21 bp, Artificial Sequence
  synthetic polynucleotides B (-304 ˜ -86) ChIP-qPCR primer R
tgatttggcgactgcatgtgc

SEQ ID NO: 28 - DNA, 20 bp, Artificial Sequence
  synthetic polynucleotides C (-870 ˜ -664) ChIP-qPCR primer F
ctccagcctgggcaacagag

SEQ ID NO: 29 - DNA, 20 bp, Artificial Sequence
  synthetic polynucleotides C (-870 ˜ -664) ChIP-qPCR primer R
ttgcttgaacctgggagggg

SEQ ID NO: 30 - DNA, 24 bp, Artificial Sequence
  synthetic polynucleotides D (-1623 ˜ -1474) ChIP-qPCR primer F
cctgcatgctaggagaaattccca

SEQ ID NO: 31 - DNA, 23 bp, Artificial Sequence
  synthetic polynucleotides D (-1623 ˜ -1474) ChIP-qPCR primer R
aggtctctgccttcattcttggc

SEQ ID NO: 32 - DNA, 30 bp, Artificial Sequence
  synthetic polynucleotides Flag-ANKRD9 cloning primer F
aaagaattcaccatgccgtgggacgcgcgg

SEQ ID NO: 33 - DNA, 32 bp, Artificial Sequence
  synthetic polynucleotides Flag-ANKRD9 cloning primer R
gcgcgtcgacgcctttgccagtgaggtccaac

SEQ ID NO: 34 - DNA, 30 bp, Artificial Sequence
  synthetic polynucleotides Flag-ASB1 cloning primer F
aaagaattcaccatggcggagggcggcagc

SEQ ID NO: 35 - DNA, 30 bp, Artificial Sequence
  synthetic polynucleotides Flag-ASB1 cloning primer R
gcgcgtcgacctcatggagtagaaacttct

SEQ ID NO: 36 - DNA, 30 bp, Artificial Sequence
  synthetic polynucleotides Flag-ASB3 cloning primer F
ccggaattcaccatggattttacagaggct

SEQ ID NO: 37 - DNA, 30 bp, Artificial Sequence
  synthetic polynucleotides Flag-ASB3 cloning primer R
gcttgtcgactccatcttgaatagctgcca

SEQ ID NO: 38 - DNA, 30 bp, Artificial Sequence
  synthetic polynucleotides Flag-ASB4 cloning primer F
attgaattcaccatggacggcaccactgcc

SEQ ID NO: 39 - DNA, 31 bp, Artificial Sequence
  synthetic polynucleotides Flag-ASB4 cloning primer R
gcgcgtcgacataaataattccctctggctc

SEQ ID NO: 40 - DNA, 30 bp, Artificial Sequence
  synthetic polynucleotides Flag-ASB5 cloning primer F
ccggaattcaccatgtcggtgttagaagaa

SEQ ID NO: 41 - DNA, 30 bp, Artificial Sequence
  synthetic polynucleotides Flag-ASB5 cloning primer R
gcgcgtcgactcgatactgtaagaaattct

SEQ ID NO: 42 - DNA, 30 bp, Artificial Sequence
  synthetic polynucleotides Flag-ASB6 cloning primer F
attgaattcaccatgccgttcctgcacggc

SEQ ID NO: 43 - DNA, 31 bp, Artificial Sequence
  synthetic polynucleotides Flag-ASB6 cloning primer R
aattgtcgacgatgtcatcttccacggagcc

SEQ ID NO: 44 - DNA, 28 bp, Artificial Sequence
  synthetic polynucleotides Flag-ASB7 cloning primer F
atgttacaccatcattgtcgaaggaacc

SEQ ID NO: 45 - DNA, 30 bp, Artificial Sequence
  synthetic polynucleotides Flag-ASB7 cloning primer R
cgcggatccgatatcatcaaatttgtgttt

SEQ ID NO: 46 - DNA, 30 bp, Artificial Sequence
  synthetic polynucleotides Flag-ASB8 cloning primer F
ccggaattcaccatgagttccagtatgtgg

SEQ ID NO: 47 - DNA, 30 bp, Artificial Sequence
  synthetic polynucleotides Flag-ASB8 cloning primer R
gcgcgtcgacttctaaaagtaacaggtatt

SEQ ID NO: 48 - DNA, 28 bp, Artificial Sequence
  synthetic polynucleotides Flag-ASB9 cloning primer F
atggatggcaaacaagggggcatggatg

SEQ ID NO: 49 - DNA, 31 bp, Artificial Sequence
  synthetic polynucleotides Flag-ASB9 cloning primer R
cgcggatccaagatgtaggagaaactgtttc

SEQ ID NO: 50 - DNA, 30 bp, Artificial Sequence
  synthetic polynucleotides Flag-ASB10 cloning primer F
gcggaattcaccatgctcatgagttggtct

SEQ ID NO: 51 - DNA, 31 bp, Artificial Sequence
  synthetic polynucleotides Flag-ASB10 cloning primer R
ccttgtcgacgtagagcacgccctcaaaatc

SEQ ID NO: 52 - DNA, 28 bp, Artificial Sequence
  synthetic polynucleotides Flag-ASB11 cloning primer F
atggaagatggtcctgttttctatggct

SEQ ID NO: 53 - DNA, 31 bp, Artificial Sequence
  synthetic polynucleotides Flag-ASB11 cloning primer R
cgcggatccttggtataggaggaatcgttcg

SEQ ID NO: 54 - DNA, 30 bp, Artificial Sequence
  synthetic polynucleotides Flag-ASB12 cloning primer F
ccggaattcaccatgagaatagttctccaa

SEQ ID NO: 55 - DNA, 30 bp, Artificial Sequence
  synthetic polynucleotides Flag-ASB12 cloning primer R
gcgcgtcgaccagttggtgttttaggtacg

SEQ ID NO: 56 - DNA, 33 bp, Artificial Sequence
  synthetic polynucleotides Flag-ASB13 cloning primer F
attgaattcaccatggagccccgggcggcggac

SEQ ID NO: 57 - DNA, 31 bp, Artificial Sequence
  synthetic polynucleotides Flag-ASB13 cloning primer R
gcgcgtcgacgttgtaggagaggtaatcaat

SEQ ID NO: 58 - DNA, 28 bp, Artificial Sequence
  synthetic polynucleotides Flag-ASB14 cloning primer F
atggataattacaccagcgatgaagaca

SEQ ID NO: 59 - DNA, 30 bp, Artificial Sequence
  synthetic polynucleotides Flag-ASB14 cloning primer R
ttgcgtcgacccaggttcctgtaaaaattc

SEQ ID NO: 60 - DNA, 28 bp, Artificial Sequence
  synthetic polynucleotides Flag-ASB15 cloning primer F
atggatactaatgatgaccctgatgaag

SEQ ID NO: 61 - DNA, 30 bp, Artificial Sequence
  synthetic polynucleotides Flag-ASB15 cloning primer R
cgcggatcctgtcaattttagctcttgtcc

SEQ ID NO: 62 - DNA, 30 bp, Artificial Sequence
  synthetic polynucleotides Flag-ASB16 cloning primer F
acggaattcaccatggcaagagagaccttc

SEQ ID NO: 63 - DNA, 30 bp, Artificial Sequence
  synthetic polynucleotides Flag-ASB16 cloning primer R
aattgtcgacctggatgcacccctccacac

SEQ ID NO: 64 - DNA, 33 bp, Artificial Sequence
  synthetic polynucleotides Flag-ASB17 cloning primer F
ccggaattcaccatgagtaaatctactaaatta

SEQ ID NO: 65 - DNA, 31 bp, Artificial Sequence
  synthetic polynucleotides Flag-ASB17 cloning primer R
gcgcgtcgacgatttctaaattcagatagtt

SEQ ID NO: 66 - DNA, 31 bp, Artificial Sequence
  synthetic polynucleotides Flag-ASB18 cloning primer F
tgaattcaccatgtccaactcggattacctt

SEQ ID NO: 67 - DNA, 30 bp, Artificial Sequence
  synthetic polynucleotides Flag-ASB18 cloning primer R
ccatgtcgacgtgcaaaacaccctgtggct

SEQ ID NO: 68 - DNA, 30 bp, Artificial Sequence
  synthetic polynucleotides Flag-CIS cloning primer F
agaattcaccatgtacctagaacacaccag

SEQ ID NO: 69 - DNA, 30 bp, Artificial Sequence
  synthetic polynucleotides Flag-CIS cloning primer R
aattgtcgacgagctggaaggggtactgtc

SEQ ID NO: 70 - DNA, 30 bp, Artificial Sequence
  synthetic polynucleotides Flag-Muf1 cloning primer F
agaattcaccatggcggcgcccgaggcctg

SEQ ID NO: 71 - DNA, 30 bp, Artificial Sequence
  synthetic polynucleotides Flag-Muf1 cloning primer R
aattgtcgaccatggtgctaacataatctg

SEQ ID NO: 72 - DNA, 30 bp, Artificial Sequence
  synthetic polynucleotides Flag-PCMTD1 cloning primer F
ttggaattcaccatgggaggagctgtgagt

SEQ ID NO: 73 - DNA, 30 bp, Artificial Sequence
  synthetic polynucleotides Flag-PCMTD1 cloning primer R
gcgcgtcgactttgtctctaaaatatgtca

SEQ ID NO: 74 - DNA, 30 bp, Artificial Sequence
  synthetic polynucleotides Flag-PCMTD2 cloning primer F
agaattcaccatgggcggtgctgtgagtgc

SEQ ID NO: 75 - DNA, 30 bp, Artificial Sequence
  synthetic polynucleotides Flag-PCMTD2 cloning primer R
aattgtcgactttttctctgtaataaagca

SEQ ID NO: 76 - DNA, 30 bp, Artificial Sequence
  synthetic polynucleotides Flag-RAB40A cloning primer F
aaagaattcaccatgagcgccccgggcagc

SEQ ID NO: 77 - DNA, 33 bp, Artificial Sequence
  synthetic polynucleotides Flag-RAB40A cloning primer R
gcgcgtcgacagaaattttgcagctgtttctgg

SEQ ID NO: 78 - DNA, 30 bp, Artificial Sequence
  synthetic polynucleotides Flag-RAB40B cloning primer F
aaagaattcaccatgagcgccctgggcagc

SEQ ID NO: 79 - DNA, 33 bp, Artificial Sequence; identical to SEQ ID NO: 77
  synthetic polynucleotides Flag-RAB40B cloning primer R

SEQ ID NO: 80 - DNA, 30 bp, Artificial Sequence
  synthetic polynucleotides Flag-RAB40C cloning primer F
ttagaattcaccatgggctcgcagggcagt

SEQ ID NO: 81 - DNA, 30 bp, Artificial Sequence
  synthetic polynucleotides Flag-RAB40C cloning primer R
ttgcgtcgacggagatcttgcagttactcc

SEQ ID NO: 82 - DNA, 30 bp, Artificial Sequence
  synthetic polynucleotides Flag-SSB1 cloning primer F
agaattcaccatgggtcagaaggtcactgg

SEQ ID NO: 83 - DNA, 30 bp, Artificial Sequence
  synthetic polynucleotides Flag-SSB1 cloning primer R
aattgtcgacctggtagaggaggtaggcct

SEQ ID NO: 84 - DNA, 30 bp, Artificial Sequence
  synthetic polynucleotides Flag-SSB2 cloning primer F
agaattcaccatgggccagacagctctggc

SEQ ID NO: 85 - DNA, 30 bp, Artificial Sequence
  synthetic polynucleotides Flag-SSB2 cloning primer R
aattgtcgacctggtagagcaggtagcgct

SEQ ID NO: 86 - DNA, 30 bp, Artificial Sequence
  synthetic polynucleotides Flag-SSB3 cloning primer F
agaattcaccatggccagacgcccccggaa

SEQ ID NO: 87 - DNA, 30 bp, Artificial Sequence
  synthetic polynucleotides Flag-SSB3 cloning primer R
aattgtcgacggtccggcggcagcgcttcc

SEQ ID NO: 88 - DNA, 30 bp, Artificial Sequence
  synthetic polynucleotides Flag-SSB4 cloning primer F
agaattcaccatgggccagaagctctcggg

SEQ ID NO: 89 - DNA, 30 bp, Artificial Sequence
  synthetic polynucleotides Flag-SSB4 cloning primer R
aattgtcgacctggtactgcagatagtttt

SEQ ID NO: 90 - DNA, 30 bp, Artificial Sequence
  synthetic polynucleotides Flag-SOCS1 cloning primer F
agaattcaccatggtagcacacaaccaggt

SEQ ID NO: 91 - DNA, 30 bp, Artificial Sequence
  synthetic polynucleotides Flag-SOCS1 cloning primer R
aattgtcgacaatctggaaggggaaggagc

SEQ ID NO: 92 - DNA, 30 bp, Artificial Sequence
  synthetic polynucleotides Flag-SOCS2 cloning primer F
agaattcaccatgaccctgcggtgccttga

SEQ ID NO: 93 - DNA, 30 bp, Artificial Sequence
  synthetic polynucleotides Flag-SOCS2 cloning primer R
aattgtcgactacctggaatttatattctt

SEQ ID NO: 94 - DNA, 30 bp, Artificial Sequence
  synthetic polynucleotides Flag-SOCS3 cloning primer F
agaattcaccatggtcacccacagcaagtt

SEQ ID NO: 95 - DNA, 30 bp, Artificial Sequence
  synthetic polynucleotides Flag-SOCS3 cloning primer R
aattgtcgacaagcggggcatcgtactggt

SEQ ID NO: 96 - DNA, 30 bp, Artificial Sequence
  synthetic polynucleotides Flag-SOCS4 cloning primer F
agaattcaccatggcagaaaataatgaaaa

SEQ ID NO: 97 - DNA, 30 bp, Artificial Sequence
  synthetic polynucleotides Flag-SOCS4 cloning primer R
aattgtcgacgcattgctgttctggtgcat

SEQ ID NO: 98 - DNA, 29 bp, Artificial Sequence
  synthetic polynucleotides Flag-SOCS5 cloning primer F
aatatcgataccatggataaagtgggaaa

SEQ ID NO: 99 - DNA, 30 bp, Artificial Sequence
  synthetic polynucleotides Flag-SOCS5 cloning primer R
aattgtcgacctttgccttgactggttctc

SEQ ID NO: 100 - DNA, 30 bp, Artificial Sequence
  synthetic polynucleotides Flag-SOCS6 cloning primer F
agaattcaccatgaagaaaattagtcttaa

SEQ ID NO: 101 - DNA, 30 bp, Artificial Sequence
  synthetic polynucleotides Flag-SOCS6 cloning primer R
aattgtcgacgtgcttctcctgtaaataat

SEQ ID NO: 102 - DNA, 33 bp, Artificial Sequence
  synthetic polynucleotides Flag-SOCS7 cloning primer F
ccggaattcaccatggtgttccgcaacgtgggt

SEQ ID NO: 103 - DNA, 31 bp, Artificial Sequence
  synthetic polynucleotides Flag-SOCS7 cloning primer R
gcgtgtcgaccgtggagggttccacctcttg

SEQ ID NO: 104 - DNA, 30 bp, Artificial Sequence
  synthetic polynucleotides Flag-TULP4 cloning primer F
tgaattcaccatgtatgcagcagtggaaca

SEQ ID NO: 105 - DNA, 30 bp, Artificial Sequence
  synthetic polynucleotides Flag-TULP4 cloning primer R
ccatgtcgactttgaggcgctgagtcacgt

SEQ ID NO: 106 - DNA, 30 bp, Artificial Sequence
  synthetic polynucleotides Flag-WSB1 cloning primer F
agaattcaccatggccagctttcccccgag

SEQ ID NO: 107 - DNA, 30 bp, Artificial Sequence
  synthetic polynucleotides Flag-WSB1 cloning primer R
aattgtcgacaatacgatacgagagaaact

SEQ ID NO: 108 - DNA, 33 bp, Artificial Sequence
  synthetic polynucleotides Flag-WSB2 cloning primer F
attgaattcaccatggaggccggagaggaaccg

SEQ ID NO: 109 - DNA, 31 bp, Artificial Sequence
  synthetic polynucleotides Flag-WSB2 cloning primer R
gcgcgtcgacaaaagtcctgtatgtgaggaa

SEQ ID NO: 110 - DNA, 30 bp, Artificial Sequence
  synthetic polynucleotides BIK cloning primer F
tgaattcaccatgtctgaagtaagacccct

SEQ ID NO: 111 - DNA, 30 bp, Artificial Sequence
  synthetic polynucleotides BIK cloning primer R
ccatgtcgactcacttgagcagcaggtgca

SEQ ID NO: 112 - DNA, 30 bp, Artificial Sequence
  synthetic polynucleotides Flag-BCL2 cloning primer F
aaagaattcaccatggcgcacgctgggaga

SEQ ID NO: 113 - DNA, 29 bp, Artificial Sequence
  synthetic polynucleotides Flag-BCL2 cloning primer R
atatgtcgaccttgtggcccagataggca

SEQ ID NO: 114 - DNA, 30 bp, Artificial Sequence
  synthetic polynucleotides 3xFlag-BIK cloning primer F
gaagatcttcatgtctgaagtaagacccct

SEQ ID NO: 115 - DNA, 29 bp, Artificial Sequence
  synthetic polynucleotides 3xFlag-BIK cloning primer R
gctctagatcacttgagcagcaggtgcag

SEQ ID NO: 116 - DNA, 28 bp, Artificial Sequence
  synthetic polynucleotides Myc-ASB11 cloning primer F
atggaagatggtcctgttttctatggct

SEQ ID NO: 117 - DNA, 31 bp, Artificial Sequence; identical to SEQ ID NO: 53
  synthetic polynucleotides Myc-ASB11 cloning primer R

SEQ ID NO: 118 - DNA, 28 bp, Artificial Sequence
  synthetic polynucleotides Myc-DNASB11 cloning primer F
atggaagatggtcctgttttctatggct

SEQ ID NO: 119 - DNA, 30 bp, Artificial Sequence
  synthetic polynucleotides Myc-DNASB11 cloning primer R
ggaggatccaggtgggccttcacggagcaa

SEQ ID NO: 120 - DNA, 30 bp, Artificial Sequence
  synthetic polynucleotides V5-ROC2 cloning primer F
tgaattcaccatggccgacgtggaagacgg

SEQ ID NO: 121 - DNA, 30 bp, Artificial Sequence
  synthetic polynucleotides V5-ROC2 cloning primer R
aattgtcgactttgccgattctttggacca

SEQ ID NO: 122 - DNA, 28 bp, Artificial Sequence
  synthetic polynucleotides Flag-DNASB11 cloning primer F
atggaagatggtcctgttttctatggct

SEQ ID NO: 123 - DNA, 30 bp, Artificial Sequence; identical to SEQ ID NO: 119
  synthetic polynucleotides Flag-DNASB11 cloning primer R

SEQ ID NO: 124 - DNA, 30 bp, Artificial Sequence
  synthetic polynucleotides Myc-XBP1(s) cloning primer F
ataggatccaccatggtggtggtggcagcc

SEQ ID NO: 125 - DNA, 30 bp, Artificial Sequence
  synthetic polynucleotides Myc-XBP1(s) cloning primer R
atatgtcgacgacactaatcagctggggaa

SEQ ID NO: 126 - DNA, 27 bp, Artificial Sequence
  synthetic polynucleotides BIK(115R) cloning primer F
ttcaccacacttagggagaacataatg

SEQ ID NO: 127 - DNA, 27 bp, Artificial Sequence
  synthetic polynucleotides BIK(115R) cloning primer R
cattatgttctccctaagtgtggtgaa

SEQ ID NO: 128 - DNA, 27 bp, Artificial Sequence
  synthetic polynucleotides BIK(160R) cloning primer F
ctgcacctgctgctcaggtgagtcgac

SEQ ID NO: 129 - DNA, 27 bp, Artificial Sequence
  synthetic polynucleotides BIK(160R) cloning primer R
gtcgactcacctgagcagcaggtgcag

SEQ ID NO: 130 - DNA, 27 bp, Artificial Sequence
  synthetic polynucleotides BIKDD cloning primer F
gttcttggcatggatgacgatgaagag

SEQ ID NO: 131 - DNA, 27 bp, Artificial Sequence
  synthetic polynucleotides BIKDD cloning primer R
ctcttcatcgtcatccatgccaagaac

SEQ ID NO: 132 - DNA, 30 bp, Artificial Sequence
  synthetic polynucleotides pLAS5W-Myc-ASB11 cloning primer F
attgctagcaccatggaagatggtcctgtt

SEQ ID NO: 133 - DNA, 30 bp, Artificial Sequence
  synthetic polynucleotides pLAS5W-Myc-ASB11 cloning primer R
ggaattcttatcacagcaggtcctcctcgc

SEQ ID NO: 134 - DNA, 33 bp, Artificial Sequence
  synthetic polynucleotides ASB11 promoter 1949 cloning primer F
gatgctagcatgttatagaacaatgtacatcac

SEQ ID NO: 135 - DNA, 33 bp, Artificial Sequence
  synthetic polynucleotides ASB11 promoter 1949 cloning primer R
atcctcgagtttggcttatgctctgtatagggt

SEQ ID NO: 136 - DNA, 31 bp, Artificial Sequence
  synthetic polynucleotides ASB11 promoter 478 cloning primer F
gatgctagcacgcgcccggccaggtaaatgg

SEQ ID NO: 137 - DNA, 33 bp, Artificial Sequence; identical to SEQ ID NO: 135
  synthetic polynucleotides ASB11 promoter 478 cloning primer R

SEQ ID NO: 138 - DNA, 30 bp, Artificial Sequence
  synthetic polynucleotides ASB11 promoter 478 dNFY cloning primer F
ctattggatgtttctgaaggaagtttcttt

SEQ ID NO: 139 - DNA, 30 bp, Artificial Sequence
  synthetic polynucleotides ASB11 promoter 478 dNFY cloning primer R
aaagaaacttccttcagaaacatccaatag

SEQ ID NO: 140 - DNA, 21 bp, Artificial Sequence
  synthetic polynucleotides Cul2 Clone #1 Target
gcaagctacatcggatgtata

SEQ ID NO: 141 - DNA, 21 bp, Artificial Sequence
  synthetic polynucleotides Cul2 Clone #2 Target
cgtttgcagttgatgtgtctt

SEQ ID NO: 142 - DNA, 21 bp, Artificial Sequence
  synthetic polynucleotides Cul5 Clone #1 Target
gcagactgaattagtagaaat

SEQ ID NO: 143 - DNA, 21 bp, Artificial Sequence
  synthetic polynucleotides Cul5 Clone #2 Target
cgctgtattgtttgcatggaa

SEQ ID NO: 144 - DNA, 21 bp, Artificial Sequence
  synthetic polynucleotides ASB11 Clone #1 Target
cagtgctgcatgtgtcaatgt

SEQ ID NO: 145 - DNA, 21 bp, Artificial Sequence
  synthetic polynucleotides ASB11 Clone #2 Target
ggatagcagaagagatctatg

SEQ ID NO: 146 - DNA, 21 bp, Artificial Sequence
  synthetic polynucleotides ATF6 Clone #1 Target
cctagtccaaagcgaagagtt

SEQ ID NO: 147 - DNA, 21 bp, Artificial Sequence
  synthetic polynucleotides ATF6 Clone #2 Target
agaactgtctcgtactagaat

SEQ ID NO: 148 - DNA, 21 bp, Artificial Sequence
  synthetic polynucleotides BIK Clone #1 Target
tcttgatggagaccctcctgt

SEQ ID NO: 149 - DNA, 21 bp, Artificial Sequence
  synthetic polynucleotides NFYB Clone #1 Target
cacaacatcatatcaacagat

SEQ ID NO: 150 - DNA, 21 bp, Artificial Sequence
  synthetic polynucleotides NFYB Clone #2 Target
ccaaagaatgtgttcaagaat

SEQ ID NO: 151 - DNA, 21 bp, Artificial Sequence
  synthetic polynucleotides NFYC Clone #1 Target
ctggctcgtattaagaagatt

SEQ ID NO: 152 - DNA, 21 bp, Artificial Sequence
  synthetic polynucleotides NFYC Clone #2 Target
atgatatcgccatggcaatta

SEQ ID NO: 153 - DNA, 21 bp, Artificial Sequence
  synthetic polynucleotides PERK Clone #1 Target
cccaaactgattataggtaac

SEQ ID NO: 154 - DNA, 21 bp, Artificial Sequence
  synthetic polynucleotides PERK Clone #2 Target
gaaacagctattctcataaag

SEQ ID NO: 155 - DNA, 21 bp, Artificial Sequence
  synthetic polynucleotides XBP1 Clone #1 Target
gcctgtctgtacttcattcaa

SEQ ID NO: 156 - DNA, 21 bp, Artificial Sequence
  synthetic polynucleotides XBP1 Clone #2 Target
gaacagcaagtggtagattta

(19) United States
(12) Patent Application Publication
(10) Pub. No.: US 2020/0215153 A1
(43) Pub. Date: Jul. 9, 2020

(54) CANCER TREATMENT AND/OR PREVENTION THROUGH REGULATION OF UBIQUITINATION

(71) Applicant: Academia Sinica, Taipei (TW)

(72) Inventors: Ruey-Hwa CHEN, Taipei (TW); Fei-Yun CHEN, Kaohsiung City (TW); Min-Yu HUANG, Pingtung City (TW)

(21) Appl. No.: 16/712,750

(22) Filed: Dec. 12, 2019

Related U.S. Application Data
(60) Provisional application No. 62/779,094, filed on Dec. 13, 2018.

(51) Int. Cl. A61K 38/17; A61K 9/51; A61K 48/00; A61P 35/00; A61K 31/381; A61K 31/11; A61K 31/37; A61K 31/438; A61K 31/473; A61K 31/519; A61K 31/4985; A61K 31/506; A61K 31/5377

(52) U.S. Cl. CPC A61K 38/17; A61K 9/51; A61K 48/0066; A61P 35/00; A61K 31/381; A61K 31/11; A61K 31/5377; A61K 31/438; A61K 31/473; A61K 31/519; A61K 31/4985; A61K 31/506; A61K 31/37

(57) ABSTRACT

This disclosure provides for a method of treating and/or preventing cancer in a subject by targeting the BIK degradation pathway in combination with the administration of an active BIKDD. Also described herein are compositions comprising an active BIKDD and methods of their making an use for the treatment of cancer.

This application claims the benefit of priority of U.S. Application No. 62/779,094, filed on Dec. 13, 2018, the content of which is incorporated herein.

### SEQUENCE LISTING

The instant application contains a Sequence Listing which has been submitted in ASCII format via EFS-Web and is hereby incorporated by reference in its entirety. Said ASCII copy, created on Mar. 19, 2020, is named G4590-04301_US8659-SEQ_ST25, and is 35 kilobytes in size.

### FIELD OF THE INVENTION

The present invention relates to a field of cancer prevention and/or treatment. Particularly, the present invention relates to CRL5^{ASB11} as a ubiquitin ligase targeting BIK for ubiquitination and proteasomal degradation and anti-cancer strategy through targeting BIK degradation pathway in combined with the administration of an active BIK.

### BACKGROUND OF THE INVENTION

The following includes information that may be useful in understanding the present invention. It is not an admission that any of the information, publications or documents specifically or implicitly referenced herein is prior art, or essential, to the presently described or claimed inventions. All publications, patents, related applications, and other written or electronic materials mentioned or identified herein are hereby incorporated herein by reference in their entirety. The information incorporated is as much a part of the application as filed as if all of the text and other content was repeated in the application, and should be treated as part of the text and content of the application as filed.

Regulation of cell death is crucial for normal cell physiology, tissue homeostasis, and development. The Bcl-2 family determines the commitment of cells to apoptotic death and consists of three subgroups: the pro-survival Bcl-2 like proteins, the multidomain pro-apoptotic BAX/BAK proteins and the pro-apoptotic BH3-only proteins. The BH3-only proteins function at the apex of Bcl-2 family-controlled apoptotic pathway and activate BAX/BAK through two distinct mechanisms. In the direct activation mechanism, certain BH3-only proteins, such as BIM and tBID, bind BAX/BAK transiently to trigger their oligomerization at the outer mitochondrial membrane, thereby inducing cytochrome C release for apoptosis induction. However, most of the BH3-only proteins act through an indirect mechanism by binding to the pro-survival Bcl-2 proteins, thereby preventing them from neutralizing BAX/BAK.

Consistent with the function of BH3-only proteins as the fulcrum of Bcl-2-family-governed apoptotic pathway, their expression and activity are tightly regulated under various physiological and stressed conditions. For instance, PUMA and NOXA are transcriptionally upregulated by p53 under DNA damage, whereas BIM expression is transcriptionally induced by CHOP during endoplasmic reticulum (ER) stress. In addition to being regulated at the transcriptional level, BH3-only proteins also undergo various posttranslational modifications. For instance, BAD and BIM are negatively regulated by Akt- and ERK-induced phosphorylation, respectively. Ubiquitin-mediated proteolysis is another mechanism to regulate the abundance of BH3-only proteins and BIM is the most well studied member undergoing such mode of regulation. BIM is ubiquitinated by SCF-PTRCP complex upon phosphorylation by RSK/ERK in the G1/S phase and by APC^{cdc20} during mitosis. Regulation of these ubiquitination pathways could influence on the sensitivity of cancer cells to anti-tumor agents.

### SUMMARY OF THE INVENTION

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

The present disclosure-identifies CRL5^{ASB11} as a ubiquitin ligase targeting BIK for ubiquitination and proteasomal degradation. Under ER stress, ASB11 (Ankyrin repeat and SOCS box protein 11) is transcriptionally activated by XBPls, an effector of IRE1-alpha. In contrast, DNA damage-induced p53 downregulates IRE1-alpha to repress ASB11. Consequently, BIK ubiquitination and degradation are enhanced by ER stress and reduced by DNA damage, thereby oppositely regulating cell life-death decision in the two stressed conditions. The present disclosure also shows that targeting BIK degradation pathway combined with the administration of an active BIK could offer an effective anti-cancer strategy.

Certain embodiments include using CRL5^{ASB11} as an ubiquitin ligase targeting BIK for ubiquitination and proteasomal degradation. In some embodiments, the ASB11 potentiates BIK ubiquitination; the depletion of ASB11 reduces BIK ubiquitination level; and the overexpression of ASB11 decreases BIK protein level and increases BIK protein degradation. In another embodiment, the ASB11 is transcriptionally activated by XBP1. In a further embodiment, the XBP1 is an effector of IRE1α.

In another aspect, the expression of ASB11 is upregulated by tunicamycin or a calcium pump inhibitor. Certain embodiment of calcium pump inhibitor is thapsigargin.

In another aspect, ER stress can promote BIK ubiquitination and degradation through XBP1-induced ASB11 upregulation.

In another aspect, a genotoxic agent acts through p53 to down-regulate IRE1α and ASB11, thereby stabilizing BIK. Certain embodiments of genotoxic agent include doxorubicin and 5-flurouracil.

In some aspects, this disclosure provides for a method for preventing and/or treating a subject having cancer, comprising administering to said subject a composition comprising an active BIK and a BIKDD ubiquitination pathway inhibitor or blocker. In some aspects, the BIKDD ubiquitination pathway is the BIK degradation pathway. In some aspects, the BIKDD ubiquitination pathway inhibitor or blocker is a ubiquitin ligase. In some aspects, the ubiquitin ligase is CRL5^{ASB11}. In some aspects, the BIKDD ubiquitination pathway further comprises ASB11. In some aspects, the depletion of ASB11 reduces BIK ubiquitination level resulting in the inhibition or blockage of the BIKDD ubiquitination pathway. In some aspects, the overexpression of ASB11 decreases BIK protein level and increases BIK protein degradation. ASB11 is transcriptionally activated by XBP1.

In some aspects, XBP1 is an effector of IRE1-alpha. In some aspects, the expression of ASB11 is upregulated by tunicamycin or a calcium pump inhibitor. In some aspects, the calcium pump inhibitor is thapsigargin.

In some aspects, ER stress promotes BIK ubiquitination and degradation through XBP1-induced ASB11 upregulation.

In some aspects, a genotoxic agent acts through p53 to down-regulate IRE1-alpha and ASB11, thereby stabilizing BIK. Genotoxic agents are selected from doxorubicin or 5-flurouracil.

In some aspects, this disclosure provides for a method for preventing and/or treating a cancer in a subject, comprising administering to said subject a combination of a BIK gene therapy and an IRE1-alpha inhibitor. The BIK gene therapy and the administration of the IRE1-alpha inhibitor can be performed separately or simultaneously. In some aspects, the BIK gene therapy comprises the administration of a BIKDD lipid nanoparticle. In some aspects, the BIK lipid nanoparticle is selected from: BIKDD, C-VISA BIKDD: liposome, CMV-BIKDD, or SV-BIKDD. In some aspects, the IRE1-alpha inhibitor is selected from: 7-Hydroxy-4-methyl-2-oxo-2H-1-benzopyran-8-carboxaldehyde 4μ8C (4-methyl umbelliferone 8-carbaldehyde), IRE1 Inhibitor I (N-[(2-Hydroxy-1-naphthalenyl)methylene]-2-thiophenesulfonamide; STF-083010), 3-ethoxy-5,6-dibromosalicylaldehyde, (R)-2-(3,4-dichlorobenzyl)-N-(4-methylbenzyl)-2,7-diazaspiro[4.5]decane-7-carboxamide (GS K2850163), 3,6-DMAD hydrochloride, Toyocamycin (Vengicide), KIRA6, APY29, Kira8 (AMG-18), MKC9989, MKC8866, or combinations thereof.

In some aspects, the method involves BIK gene therapy which comprises administering a BIK lipid nanoparticle with an inhibitory RNA for IRE1-alpha. In some aspects, the inhibitory RNA for IRE1-alpha is selected from an interfering RNA, shRNA, siRNA, ribozymes, or antisense oligonucleotide for IRE1-alpha. In some aspects, the inhibitory RNA for IRE-alpha is delivered with a viral vector. In some aspects, the viral vector is adenovirus vector.

In some aspects, the method involves the treatment of cancer, wherein the cancer is selected from: neuroblastoma; lung cancer; bile duct cancer; non-small cell lung carcinoma; hepatocellular carcinoma; head and neck squamous cell carcinoma; squamous cell cervical carcinoma; lymphoma; nasopharyngeal carcinoma; gastric cancer; colon cancer; uterine cervical carcinoma; gall bladder cancer; prostate cancer; breast cancer; testicular germ cell tumors; colorectal cancer; glioma; thyroid cancer; basal cell carcinoma; gastrointestinal stromal cancer; hepatoblastoma; endometrial cancer; ovarian cancer; pancreatic cancer; renal cell cancer, Kaposi's sarcoma, chronic leukemia, sarcoma, rectal cancer, throat cancer, melanoma, colon cancer, bladder cancer, mastocytoma, mammary carcinoma, mammary adenocarcinoma, pharyngeal squamous cell carcinoma, testicular cancer, gastrointestinal cancer, or stomach cancer or urothelial cancer. In some aspects, the cancer is a breast cancer. In some aspects, the cancer is a drug-resistant cancer. In some aspects, the cancer is a triple-negative breast cancer (TNBC).

### BRIEF DESCRIPTION OF THE DRAWINGS

FIG. 1. Cul5ASB11 targets BIK for ubiquitination. (a, b) Western blot analysis of endogenous BIK expression in 293T cells transiently transfected with indicated Cullin dominant negative (DN) mutants (a) or transduced with lentivirus carrying indicated shRNAs (b). (c) Immunoprecipitation analysis of the interaction between endogenous ASB11 and endogenous BIK in 293T cells. (d) In vitro interaction of ASB11 with BIK. Purified ASB11 bound on anti-Myc beads was incubated with BIK separately purified by and eluted from anti-Flag beads. The bound proteins were analyzed by Western blot. (e) Analysis of BIK ubiquitination in 293T cells transfected with indicted constructs. The ubiquitinated proteins were pulled down under denaturing conditions by Ni-NTA agarose and analyzed by Western blot. (f) Analysis of BIK ubiquitination in 293T cells stably expressing control or ASB11 shRNA and transfected with indicated constructs. The knockdown efficiency of ASB11 shRNAs are shown in FIG. 2d. (g) In vitro ubiquitination assay for BIK. Flag-BIK purified from 293T cells was incubated with E1, E2, His-ubiquitin, and/or ASB11-based Cul5 complex purified from transfected cells. The integrity of input E3 ligase complex was shown on the right.

FIG. 2. ASB11 promotes BIK proteasomal degradation. (a) Western blot analysis of endogenous BIK in 293T cells transfected with indicated ASB11 constructs. (b, c) Western blot analysis of BIK levels in 293T cells transfected with ASB11 and treated with indicated agents. (d) Western blot analysis of BIK levels in indicated cells stably expressing control or ASB11 shRNAs. (e) Western blot analysis of BIK in 293T derivatives as in (d) treated with cycloheximide for indicated time points.

FIG. 3. ER stress induces ASB11 transcription through XBP1. (a, b) RT-qPCR analysis of ASB11 mRNA level in 293T cells (a) or 293T cells stably expressing control or XBP1 shRNAs (b) treated with tunicamycin or thapsigargin for 16 h. (c) RT-qPCR analysis of ASB11 mRNA expression in 293T cells transfected with control vector or XBP1s. (d) Schematic representation of the 5′-regulatory region of ASB11 gene, the luciferase reporters and the ChIP primers used in this study. (e, g) Luciferase reporter assay of 293T cells transfected with control or XBP1s expression construct together with the indicated reporter constructs. (f, k) qChIP assays in 293T cells (f) or 293T derivatives (k) treated with 10 μg/ml tunicamycin for 16 h using control IgG or XBP1s antibody for immunoprecipitation and indicated sets of primers for qPCR. (h) Immunoprecipitation analysis of the interaction between endogenous XBP1s and each NF-Y complex component in 293T cells treated with 10 μg/ml tunicamycin for 4 h. (i, j) Luciferase reporter assay of 293T cells stably expressing indicated shRNAs and transfected with indicated constructs or treated with 10 μg/ml tunicamycin for 16 h. The knockdown efficiencies of indicated shRNAs are shown on the right. Data in (a), (b), (c), (e), (f), (g), (i), (j), and (k) are mean±s.d., n=3. P values are determined by t-test (a, b right panel, c, e) or 1-way ANOVA with Tukey's post test (b left panel, f, g, i, j, k). **P<0.01; ***P<0.001.

FIG. 4. ER stress stimulates BIK ubiquitination and degradation through ASB11 and p97. (a) Western blot analysis of BIK and ASB11 levels in 293T cells treated with 10 μg/ml tunicamycin or 200 nM of thapsigargin for 16 h. (b) Western blot analysis of BIK levels in 293T cells treated with 10 μg/ml tunicamycin for 12 h and then with 50 μg/ml cycloheximide for indicated time periods. The relative amounts of BIK are indicated on the bottom. (c, e) Analysis of endogenous (c) or exogenous (e) BIK ubiquitination in 293T cells (c) or 293T derivatives as in FIG. 2d (e) transfected with indicated constructs and treated with tunicamycin. (d) Western blot analysis of 293T derivatives as in FIG. 2d treated with 10 μg/ml tunicamycin for 16 h. (f) Western blot analysis of BIK levels in 293T derivatives co-treated with 10 μg/ml tunicamycin and MG132 for 16 h. (g) Western blot analysis of BIK expression in 293T cells co-treated with tunicamycin and 10 μM CB-5083 for 16 h. (h, i) Western blot analysis of BIK expression in 293T cells stably expressing indicated shRNAs and treated with 10 μg/ml tunicamycin for 16 h. The knockdown efficiency of each shRNA is shown on the right. (j) Immunoprecipitation analysis of p97 interaction with ubiquitinated BIK in 293T cells treated with 10 μg/ml tunicamycin for 16 h.

FIG. 5. DNA damage-induced p53 suppresses XBP1 and ASB11 to stabilize BIK. (a) RT-qPCR analysis of ASB11 mRNA expression in indicated HCT116 cells treated with indicated dosages of doxorubicin or 5-FU for 24 h. Data are mean±s.d., *P<0.05, ***P<0.001 by t-test, n=3. (b) Western blot analysis of BIK and ASB11 expression in indicated HCT116 cells treated as in (a). (c) HCT116 p53^{+/+} cells stably expressing control or ASB11 shRNA were treated with 10 μM 5-FU for 24 h and then with cycloheximide for indicated time points. Cells were lysed for Western blot analysis of BIK expression and the relative amounts of BIK are indicated on the bottom. The expression levels of ASB11 and BIK in the stable lines are shown on the bottom panel. (d) Western blot analysis of IRE1α expression and RT-qPCR analysis of XBP1 mRNA splicing in indicated HCT116 cells treated with 3 μg/ml doxorubicin or 10 μM 5-FU for 24 h. (e) RT-qPCR analysis of ASB11 and XBPls mRNA expression in HCT116 cells transfected with XBP1s and/or treated with 3 μg/ml doxorubicin for 16 h. Data are mean±s.d., ***P<0.001 by t-test, n=3. (f) Analysis of BIK ubiquitination in HCT116 cells transfected with indicated constructs and/or treated with 3 μg/ml doxorubicin for 16 h.

FIG. 6. Regulation of ASB11-mediated BIK ubiquitination influences on cell life-death decision under ER stress and DNA damage. (a, b) ELISA assay of cell apoptosis (a) and Western blot analysis of active caspase 3 and cleaved PARP (b) in HCT116 p53^{+/+} cells stably expressing indicated ASB11 constructs and treated with 3 μg/ml doxorubicin for 24 h. (c, d) ELISA assay of cell apoptosis (c) and Western blot analysis of active caspase 3 and cleaved PARP (d) in HCT116 p53^{+/+} cells transiently transfected with ASB11 and/or BIK and treated with 3 μg/ml doxorubicin for 24 h. (e, f) ELISA assay for cell apoptosis (e) and Western blot analysis of active caspase 3 and cleaved PARP (f) in 293T cells stably expressing indicated shRNAs and treated with g/ml tunicamycin for 12 h. The knockdown efficiencies of various shRNAs are shown on the bottom. (g) Western blot analysis of BIK level in 293T cells transiently transfected with indicated BIK constructs and treated with 10 μg/ml tunicamycin for 24 h. The equal expression of BIK and BIK(2KR) in untreated cells (by adjusting the amount of plasmid used for transfection) is shown. (h, i) ELISA assay of apoptotic cells (h) and Western blot analysis of active caspase 3 and cleaved PARP (i) in 293T cells transfected using the same conditions as in (g) and treated with 10 μg/ml tunicamycin for 16 h. Data in (a), (c), (e), (h) are mean±s.d., ***P<0.001 by 1-way ANOVA with Turkey's post test, n=3. Asterisks in (b) and (d) denote a nonspecific band.

FIG. 7. IRE1α inhibitor enhances the tumor-killing effect of BIKDD. (a, c) MTT assay for the viability of indicated TNBC cells transfected with 0.5 CMV-BIKDD (a) or VISA-BIKDD (c) and treated with 10 or 100 μM STF-083010 for 48 h. CI values are indicated. (b) Western blot analysis of cleaved PARP in indicated TNBC cells transfected with CMV-BIKDD and treated with 100 μM STF-083010 for 36 h. (d) Mice orthotopically implanted with Hs578T cells carrying BIKDD or control vector and treated with STF-083010 or DMSO (see Materials and Methods). Tumor volume were measured at indicated days and plotted. Data are mean±s.d., *P<0.05; ***P<0.001 by two-way ANOVA with Turkey's post test, n=5. Tumors were surgically removed at day 49 and their sizes are shown on the right. (e) Mice orthotopically implanted with Hs578T cells and treated with VISA-BIKDD lipid nanoparticle together with STF-083010 or DMSO starting at day 28 after tumor cell implantation (see Materials and Methods). Tumor volume were measured at indicated days and plotted. Data are mean±s.d., ***P<0.001 by two-way ANOVA with Turkey's post test, n=5. Tumors were surgically removed at day 66 and their sizes are shown on the right.

### DETAILED DESCRIPTION OF THE INVENTION

The entire document is intended to be related as a unified disclosure, and it should be understood that all combinations of features described herein are contemplated, even if the combination of features are not found together in the same sentence, or paragraph, or section of this document. The present disclosure as illustratively described in the following may suitably be practiced in the absence of any element or elements, limitation or limitations, not specifically disclosed herein.

Technical terms are used by their common sense unless indicated otherwise. If a specific meaning is conveyed to certain terms, definitions of terms will be given in the following in the context of which the terms are used.

The singular forms “a”, “an”, and “the” may refer to plural articles unless specifically stated otherwise.

The terms “inhibiting” or “reducing” or any variation of these terms includes any measurable decrease or complete inhibition to achieve a desired result. The terms “promote” or “increase” or any variation of these terms includes any measurable increase or production of a protein or molecule to achieve a desired result.

The term “effective amount” is the amount necessary to achieve a specific effect, in accordance with what one of ordinary skill in the art would be readily able to determine through routine experimentation. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, e.g., by assessing the time to disease progression and/or determining the response rate.

The term “preventing” or any variation of this term means to slow, stop, or reverse progression toward a result. The prevention may be any slowing of the progression toward the result.

The terms “treatment” and “treating” embrace both preventative, i.e. prophylactic, or therapeutic, i.e. curative and/or palliative, treatment. Thus the terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus, the compounds, compositions and methods of the present invention may be used for instance as therapeutic treatment over a period of time as well as for chronic therapy. In some embodiments, the term “treatment” and “treating” refers to the therapeutic treatment.

The terms “disease” or “disorder” are used interchangeably herein, and refer to any alteration in state of the body or of some of the organs, interrupting or disturbing the performance of the functions and/or causing symptoms such as discomfort, dysfunction, distress, or even death to the person afflicted or those in contact with a person.

The term “Administering” means providing a pharmaceutical agent or composition to a subject.

BIK is the founding member of BH3-only proteins. In addition to the BH3 domain, BIK contains a transmembrane domain at its C-terminus and is mainly localized to the membrane of ER. BIK facilitates the release of ER Ca²+ store through a BAX/BAK-dependent manner. The released Ca²+ is transferred to mitochondria via ER-mitochondria contact sites, thereby activating dynamin-related GTPase DRP1 for mitochondrial cristae remodeling and cytochrome C release. BIK also increases ER-associated BAK and disrupts the interaction between Bcl-2 and inositol 1, 4, 5 triphosphate receptor, both of which contribute to the Ca²+ release from ER. Similar to several other BH3-only members, BIK is transcriptionally upregulated by p53 and E2F (Real, P. J. et al. Transcriptional activation of the proapoptotic bik gene by E2F proteins in cancer cells. FEBS Lett 580, 5905-5909 (2006)). In addition, BIK is a labile protein and can be stabilized by proteasome inhibitor (Li, C., Li, R., Grandis, J. R. & Johnson, D. E. Bortezomib induces apoptosis via Bim and Bik up-regulation and synergizes with cisplatin in the killing of head and neck squamous cell carcinoma cells. Mol Cancer Ther 7, 1647-1655 (2008)). Although such finding indicates that BIK is regulated by ubiquitination, the ubiquitin ligase responsible for this regulation has not been identified.

The unfolded protein response (UPR) is a cellular adaptive program aimed for restoring ER homeostasis under various ER stressed conditions. UPR is activated by the accumulation of misfolded proteins in the ER lumen and is mediated by three ER membrane-localized stress sensing proteins, including inositol-requiring enzyme 1 (IRE1), activating transcription factor 6 (ATF6), and protein kinase RNA-like ER kinase (PERK). The outcome of UPR can be pro-survival or pro-apoptosis depending on the strength and duration of ER stress. Under transient and mild stress conditions, UPR promotes cell survival by increasing protein folding or degradation and inhibiting protein synthesis. Under chronic ER stress, UPR facilitates apoptosis by altering the expression and/or activity of a set of pro-apoptotic regulators, including several Bcl-2 family proteins (Rodriguez, D., Rojas-Rivera, D. & Hetz, C. Integrating stress signals at the endoplasmic reticulum: The BCL-2 protein family rheostat. Biochim Biophys Acta 1813, 564-574 (2011)). However, whether UPR also regulates Bcl-2 family proteins to prevent apoptosis during the adaptive phase and how UPR switches from adaptive to apoptotic phase remain incompletely understood.

In one aspect, the present disclosure provides a method for preventing and/or treating a cancer, comprising administering an active BIK (BIKDD) in combined with the blockage of BIKDD ubiquitination pathway in a subject. In one embodiment, CRL5^{ASB11} is used as an ubiquitin ligase targeting BIK for ubiquitination and proteasomal degradation. BIK is BCL-2 interacting killer, a 160 amino acid protein which comprises a trans-membrane domain and a BH3 domain (G. Chinnadurni, Oncogene, 27 (Suppl) S20-S29 (2008)).

In one embodiment, the ASB11 potentiates BIK ubiquitination. In one embodiment, the depletion of ASB11 reduces BIK ubiquitination level. In one embodiment, the overexpression of ASB11 decreases BIK protein level and increases BIK protein degradation.

In one embodiment, the ASB11 is transcriptionally activated by a XBP1. In one embodiment, the XBP1 is an effector of IRE1α. In one embodiment, the expression of ASB11 is upregulated by tunicamycin or a calcium pump inhibitor. In one embodiment, the calcium pump inhibitor is thapsigargin. In one embodiment, a genotoxic agent acts through p53 to down-regulate IRE1α and ASB11, thereby stabilizing BIK.

In one embodiment, ER stress can promote BIK ubiquitination and degradation through XBP1-induced ASB11 upregulation.

In one embodiment, the present disclosure provides a method for preventing and/or treating a cancer, comprising a combinatory treatment of BIK gene therapy followed by an administration of an IRE1-alpha inhibitor. In some embodiments, the IRE1-alpha inhibitor can include or exclude: STF-083010, 4μ8c (4-methyl umbelliferone 8-carbaldehyde), 3,6-DMAD hydrochloride, Toyocamycin (Vengicide), KIRA6, APY29, GSK2850163, Kira8 (AMG-18), MKC9989, MKC8866, or combinations thereof. In some embodiments, the IRE1-alpha inhibitor is STF-083010.

In one embodiment, the BIK gene therapy comprises the administration of a composition comprising a BIKDD lipid nanoparticle to a subject. In one embodiment, the BIK lipid nanoparticle is selected from: BIKDD, C-VISA BIKDD: liposome, CMV-BIKDD, or SV-BIKDD. VISA is VP16-GAL4-WPRE integrated systemic amplifier gene. In one embodiment, the BIK gene therapy is conducted by performing the administration of an BIKDD lipid nanoparticle in combination with an inhibitory RNA for IRE1α. In some embodiments, the inhibitory RNA for IRE1-alpha is selected from: interfering RNA, shRNA, siRNA, temporal RNA (stRNA), ribozymes, and antisense oligonucleotides. In one embodiment, the inhibitory RNA is administered with a viral vector. In some embodiments, the inhibitory RNA is administered with direct transfection of siRNA/plasmid, or retroviral and lentiviral systems. In some embodiments, the BIKDD lipid nanoparticle is a DOTAP:cholesterol liposome comprising a vector encoding the BIKDD gene (Templeton, N. et al, Nature Biotech. 15, 647-652 (1997); Li, L., et al., Oncogene, 30, 1773-1783 (2011), each of which is hereby incorporated by reference for their description of the BIKDD lipid nanoparticle and methods of making the same). DOTAP is the lipid 1,2-dioleoyl-3-trimethylammonium-propane.

In some embodiments, the vector encoding the BIKDD gene further comprises a promoter region. In some embodiments, the promoter region is the alpha-ferroprotein (AFP) gene. In some embodiments, the dose of the BIKDD lipid nanoparticle ranges from about 0.04 mg/kg to about 10.0 mg/kg. In some embodiments, BIKDD lipid nanoparticle can be administered once, twice, or three times per day. In some embodiments, the BIKDD lipid nanoparticle can be administered over the course of 1 day, 2 days, 3, days, 4 days, 5 days, 6 days, 7 days, 2 weeks, 3 weeks, 4 weeks, 5 weeks, 6 weeks, 7 weeks, 8 weeks, 1 month, 2 months, 3 months, 4 months, 5 months, 6 months, or more.

In one embodiment, the IRE1 inhibitor is 7-Hydroxy-4-methyl-2-oxo-2H-1-benzopyran-8-carboxaldehyde (4μ8C, IRE1 Inhibitor I (N-[(2-Hydroxy-1-naphthalenyl)methylene]-2-thiophenesulfonamide; STF-083010), 3-ethoxy-5,6-dibromosalicylaldehyde, or (R)-2-(3,4-dichlorobenzyl)-N-(4-methylbenzyl)-2,7-diazaspiro[4.5]decane-7-carboxamide (GSK2850163).

The term “cancer” refers to, or describes, the physiological condition in mammals that is typically characterized by unregulated cell growth and/or hyperproliferative activities. A “tumor” comprises one or more cancerous cells. Examples of cancer include, but are not limited to, carcinoma, lymphoma, blastoma, sarcoma, and leukemia or lymphoid malignancies. In one embodiment, the cancer is a solid tumor. More particular examples of such cancers include breast cancer, cervical cancer, ovarian cancer, bladder cancer, endometrial or uterine carcinoma, prostate cancer, glioma and other brain or spinal cord cancers, squamous cell cancer (e.g., epithelial squamous cell cancer), lung cancer, including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, hepatoma, colon cancer, rectal cancer, colorectal cancer, salivary gland carcinoma, kidney or renal cancer, vulval cancer, thyroid cancer, hepatic carcinoma, anal carcinoma, penile carcinoma, as well as head and neck cancer. In one embodiment, the treatment comprises treatment of solid tumors. In one embodiment, the tumors comprises sarcomas, carcinomas or lymphomas.

In one embodiment, the cancers can include or exclude: neuroblastoma; lung cancer; bile duct cancer; non-small cell lung carcinoma; hepatocellular carcinoma; head and neck squamous cell carcinoma; squamous cell cervical carcinoma; lymphoma; nasopharyngeal carcinoma; gastric cancer; colon cancer; uterine cervical carcinoma; gall bladder cancer; prostate cancer; breast cancer; testicular germ cell tumors; colorectal cancer; glioma; thyroid cancer; basal cell carcinoma; gastrointestinal stromal cancer; hepatoblastoma; endometrial cancer; ovarian cancer; pancreatic cancer; renal cell cancer, Kaposi's sarcoma, chronic leukemia, sarcoma, rectal cancer, throat cancer, melanoma, colon cancer, bladder cancer, mastocytoma, mammary carcinoma, mammary adenocarcinoma, pharyngeal squamous cell carcinoma, testicular cancer, gastrointestinal cancer, or stomach cancer and urothelial cancer. In a further embodiment, the cancer is a breast cancer. In one embodiment, the cancer is a drug-resistant cancer. In a further embodiment, the cancer is a triple-negative breast cancer (TNBC).

The subject or individual as referred to herein and throughout the specification includes mammals. In some embodiments, the mammals are selected from: murine (specifically mice and rats), bovine, and primates. In some embodiments, the primates are human.

Administration of Compositions of this Disclosure

The compositions of the invention may be administered by any route appropriate to the condition to be treated. Suitable routes include oral, parenteral (including subcutaneous, intramuscular, intravenous, intraarterial, intradermal, intrathecal and epidural), intraperitoneal (IP), transdermal, rectal, nasal, topical (including buccal and sublingual), vaginal, intrapulmonary and intranasal. For local treatment, the compounds may be administered by intratumor administration, including perfusing or otherwise contacting the tumor with the inhibitor. It will be appreciated that the preferred route may vary with, e.g., the condition of the recipient. Where the compound is administered orally, it may be formulated as a pill, capsule, tablet, etc., with a pharmaceutically acceptable carrier or excipient. Where the compound is administered parenterally, it may be formulated with a pharmaceutically acceptable parenteral vehicle, and in a unit dosage injectable form, as detailed below.

A dose to treat human patients may range from about 1 mg to about 1000 mg of the compositions of this invention, which include an active BIK (BIKDD). The dose may be from about 1 mg, 2 mg, 2.5 mg, 4 mg, 5 mg, 7.5 mg, 10 mg, 12.5 mg, 15 mg, 17, 5 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg of an active BIK (BIKDD), or any dose ranging between any two of those doses.

A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. A typical dose when administered orally, the pill, capsule, or tablet may be ingested daily or less frequently for a specified period of time. The regimen may be repeated for a number of cycles of therapy.

Methods of Treatment with Compositions of the Invention

Compositions of the present invention are useful for treating hyperproliferative diseases, conditions and/or disorders including, but not limited to, cancer. Accordingly, an aspect of this invention includes methods of treating, or preventing, diseases or conditions that can be treated or prevented by blocking or inhibiting the BIKDD ubiquitination pathway. In one embodiment, the method comprises administering to a subject, in need thereof, a therapeutically effective amount of a composition of this invention. In one embodiment, a human patient is treated with a BIKDD lipid nanoparticle and a pharmaceutically acceptable carrier, adjuvant, or vehicle, wherein said BIKDD lipid nanoparticle is present in an amount to treat cancer and/or detectably inhibit or block the BIKDD ubiquitination pathway.

Another aspect of this invention provides a composition of this invention for use in the treatment of the diseases or conditions described herein in a subject, e.g., a human, suffering from such disease or condition. Also provided is the use of a composition of this invention in the preparation of a medicament for the treatment of the diseases and conditions described herein in a warm-blooded animal, such as a mammal, e.g. a human, suffering from such disorder.

Pharmaceutical Formulation/Compositions and Uses

In order to use a composition of this invention for the therapeutic treatment (including prophylactic treatment) of mammals including humans, it is normally formulated in accordance with standard pharmaceutical practice as a pharmaceutical composition. According to this aspect of the invention, there is provided a pharmaceutical composition comprising a BIKDD of this invention in association with a pharmaceutically acceptable diluent or carrier.

A typical formulation is prepared by mixing a BIKDD and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament). Such formulations or compositions comprise a pharmaceutically acceptable diluent, carrier, salt or adjuvant. The agent or inhibitor describe herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

In the preparation of pharmaceutical formulations or compositions as disclosed herein, either alone or complexed to targeting moieties of the present invention in the form of dosage units for oral administration the agent or inhibitor describe herein selected may be mixed with solid, powdered ingredients, such as lactose, saccharose, sorbitol, mannitol, starch, arnylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or pressed into tablets.

Soft gelatin capsules may be prepared with capsules containing a mixture of the agent or inhibitor describe herein in vegetable oil, fat, or other suitable vehicle for soft gelatin capsules. Hard gelatin capsules may contain granules of the agent or inhibitor describe herein.

Liquid preparations for oral administration may be prepared in the form of syrups, solutions or suspensions. If desired, such liquid preparations may contain coloring agents, flavoring agents, saccharin and carboxymethyl cellulose or other thickening agents. Liquid preparations for oral administration may also be prepared in the form of a dry powder to be reconstituted with a suitable solvent prior to use. Solutions for parenteral administration may be prepared as a solution of the agent or inhibitor describe herein in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

Pharmaceutical formulations of the compounds of the present invention may be prepared for various routes and types of administration. For example, a composition comprising a BIKDD having the desired degree of purity may optionally be mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compositions of this invention for use herein are preferably sterile. In particular, formulations to be used for in vivo administration must be sterile. Such sterilization is readily accomplished by filtration through sterile filtration membranes.

The pharmaceutical compositions of the invention comprising BIKDD will be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. In addition to the compounds and salt forms provided herein, the invention includes pharmaceutical compositions, including tablets, capsules, solutions, and suspensions for parenteral and oral delivery forms and formulations, comprising a pharmaceutically acceptable carrier and therapeutically effective amounts of one or more of the BIKDD ubiquitination pathway inhibitors and/or blockers herein provided.

The technology described herein is further illustrated by the following examples which in no way should be construed as being further limiting, the contents of all cited references, including literature references, issued patents, published patent applications, and co-pending patent applications, cited throughout this application are hereby expressly incorporated by reference. The technology described herein has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention.

In some embodiments, this disclosure provides:

A1. A method for preventing and/or treating a cancer, comprising administering an active BIK (BIKDD) in combined with the blockage of BIKDD ubiquitination pathway in a subject.

A2. The method of A1, wherein in the pathway, CRL5^{ASB11} is used as an ubiquitin ligase targeting BIK for ubiquitination and proteasomal degradation.

A3. The method of A1, wherein in the pathway, the ASB11 potentiates BIK ubiquitination.

A4. The method of A1, wherein in the pathway, the depletion of ASB11 reduces BIK ubiquitination level.

A5. The method of A1, wherein in the pathway, the overexpression of ASB11 decreases BIK protein level and increases BIK protein degradation.

A6. The method of A1, wherein in the pathway, the ASB11 is transcriptionally activated by XBP1.

A7. The method of A6, wherein the XBP1 is an effector of IRE1-alpha.

A8. The method of A1, wherein in the pathway, the expression of ASB11 is upregulated by tunicamycin or a calcium pump inhibitor.

A9. The method of A8, wherein the calcium pump inhibitor is thapsigargin.

A10. The method of A1, wherein in the pathway, ER stress can promote BIK ubiquitination and degradation through XBP1-induced ASB11 upregulation.

All. The method of A1, wherein a genotoxic agent acts through p53 to down-regulate IRE1-alpha and ASB11, thereby stabilizing BIK.

A12. The method of A11, wherein in the pathway, the genotoxic agents are doxorubicin and 5-flurouracil.

A13. A method for preventing and/or treating a cancer in a subject, comprising a combinatory treatment of BIK gene therapy followed by an administration of an IRE1-alpha inhibitor.

A14. The method of A13, wherein the BIK gene therapy is delivery of BIKDD lipid nanoparticle.

A15. The method of A14, wherein the BIK lipid nanoparticle is BIKDD, C-VISA BIKDD: liposome or SV-BIKDD.

A16. The method of A13, wherein the IRE1-alpha inhibitor is 7-Hydroxy-4-methyl-2-oxo-2H-1-benzopyran-8-carboxaldehyde (4μ8C, IRE1 Inhibitor I (N-[(2-Hydroxy-1-naphthalenyl)methylene]-2-thiophenesulfonamide; STF-083010), 3-ethoxy-5,6-dibromosalicylaldehyde, or (R)-2-(3,4-dichlorobenzyl)-N-(4-methylbenzyl)-2,7-diazaspiro[4.5]decane-7-carboxamide (GSK2850163).

A17. The method of A1 or A13, wherein the cancer is neuroblastoma; lung cancer; bile duct cancer; non-small cell lung carcinoma; hepatocellular carcinoma; head and neck squamous cell carcinoma; squamous cell cervical carcinoma; lymphoma; nasopharyngeal carcinoma; gastric cancer; colon cancer; uterine cervical carcinoma; gall bladder cancer; prostate cancer; breast cancer; testicular germ cell tumors; colorectal cancer; glioma; thyroid cancer; basal cell carcinoma; gastrointestinal stromal cancer; hepatoblastoma; endometrial cancer; ovarian cancer; pancreatic cancer; renal cell cancer, Kaposi's sarcoma, chronic leukemia, sarcoma, rectal cancer, throat cancer, melanoma, colon cancer, bladder cancer, mastocytoma, mammary carcinoma, mammary adenocarcinoma, pharyngeal squamous cell carcinoma, testicular cancer, gastrointestinal cancer, or stomach cancer or urothelial cancer.

A18. The method of A1 or A13, wherein the cancer is a breast cancer.

A19. The method of A1 or A13, wherein the cancer is a drug-resistant cancer.

A20. The method of A1 or A13, wherein the cancer is a triple-negative breast cancer (TNBC).

A21. The method of A1 or A13, wherein the BIK gene therapy of agent is conducted by performing administration of a BIK lipid nanoparticle in combination with an inhibitory RNA for IRE1-alpha.

A22. The method of A21, wherein the inhibitory RNA for IRE1-alpha is an interfering RNA, shRNA, siRNA, ribozymes, or antisense oligonucleotide for IRE1-alpha.

A23. The method of A13, wherein the inhibitory RNA for IRE1-alpha is delivered with a viral vector.

AA24. The method of A23, wherein the viral vector is adenovirus vector.

### EXAMPLES

Materials and Methods

Antibodies and Reagents

The ASB11 antiserum was generated and affinity purified by LTK BioLaboratories Inc., Taiwan, using the peptide TDYGANLKRRNAQGKSAL (corresponding to amino acid 248 to 265 of ASB11) as an antigen. Other antibodies used in this study are described in Supplementary Table 1. Cycloheximide, doxorubicin, 5-FU, 4μ8C, STF-083010, tunicamycin, and thapsigargin were purchased from Sigma. MG132 was obtained from Calbiochem, whereas CB-5083 was from Cayman Chemical.

Supplementary Table 1: information for antibodies uw-ii in this study

| Target | Assay | Source |
|---|---|---|
| 6xHis | WB | 631212, Clontech |
| BIK | WB | sc-1710, sc-365625, Santa Cruz |
| Cleaved Caspase-3 | WB | 9661, Cell Signaling |
| Cleaved PARP | WB | 5625, Cell Signaling |
| Cul1 | WB | 612040, BD Biosciences |
| Cul3 | WB | EPR3196Y, GeneTex |
| Flag | WB, IP | F1804, Sigma, GTX115043, GeneTex |
| GAPDH | WB | 2251-1, Epomics; GTX100118, GeneTex |
| goat IgG (HRP) | WB | GTX228416-01; GTX628547-01, GeneTex |
| HA | WB | H9658, Sigma |
| IRE1α | WB | 3294, Cell Signaling |
| mouse IgG (HRP) | WB | NA931-1ML, GE Healthcare |
| Myc | WB | sc-40, Santa Cruz; GTX29106, GeneTex |
| NFYA | WB, IP | ab6558, abcam; sc-17753, Santa Cruz |
| NFYB | WB, IP | ab6559, abcam; sc-376546, Santa Cruz |
| NFYC | WB, IP | ab232909, abcam; H00004802-M01, abnova |
| NPL4 | WB, IP | 13409, Cell Signaling |
| P53 | WB | sc-126, Santa Cruz |
| rabbit IgG | IP, ChIP | ab171870, abcam |
| rabbit IgG (HRP) | WB | NA934-1ML, GE Healthcare |
| T7 | WB | 69522, Novagen |
| Tubulin | WB | 05-829, EMD Millipore; GTX628802, GeneTex |
| UFD1 | WB, IP | 13789, Cell Signaling |
| V5 | WB | AB3792, EMD Millipore |
| VCP | WB, IP | ab11433, abcam |
| XBP1s | WB, ChIP | 619502, BioLegend |

Cell Culture and Transfection

293T, 293FT, H1299, Hs578T, MDA-MB157, and MDA-MB468 cells were maintained in Dulbecco's modified Eagle's medium (DMEM), supplemented with 10% fetal calf serum (FCS), 100 unit/ml penicillin, and 100 μg/ml streptomycin. HCT116 cells were cultured in RPMI-1640 with 10% FCS, 100 unit/ml penicillin, and 100 μg/ml streptomycin. Transfection was performed using the calcium phosphate method or the Lipofectamine 3000 reagent.

Plasmids

All Cullin DN mutant constructs were generated by site-directed mutagenesis from WT constructs obtained from Hsueh-Chi Sherry Yen. BIK cDNA was subcloned to 3× Flag-pCMV7.1.2 vector or pRK5, and BIK mutants were generated by site-directed mutagenesis. XBPls was PCR amplified from cDNAs derived from tunicamycin-treated HeLa cells and cloned to pRK5. Among the 39 Cul5 substrate adaptors, cDNA for SSB2 was provided by Soichi Miwa, RAB40A and RAB40C by John H, Brumell, RAB40B by Jorge E. Galan, WSB2 by Yue Xiong, CIS by Lu-Hai Wang, SSB1, SSB3, and SSB4 by Guan Wu, ASB1, ASB6, ASB7, and ASB12 by Yasuhiko Masuho, and ASB3, WSB1, PCMTD2, and Mufl by Joan Conaway. Other substrate adaptors were amplified from mRNA derived from 293T cells. The primers used for amplifying these cDNAs are listed in Supplementary Table 2. All substrate adaptors were cloned to pRK5-Flag. HA-ElonginB and T7-ElonginC were provided from Dong Xie, ROC2 cDNA was provided from Yue Xiong. His-ubiquitin was described previously (Yuan, W. C. et al. A Cullin3-KLHL20 Ubiquitin ligase-dependent pathway targets PML to potentiate HIF-1 signaling and prostate cancer progression. Cancer Cell 20, 214-228 (2011)). p53 cDNA was obtained from Sheau-Yann Shieh and subcloned to pRK5. CMV-BIKDD and VISA BIKDD were described previously (Lang, J. Y. et al. BIKDD eliminates breast cancer initiating cells and synergizes with lapatinib for breast cancer treatment. Cancer Cell 20, 341-356 (2011)).

SUPPLEMENTARY TABLE 2
Primers for (quantitative) PCR and cloning

| Name | Assay | Sequence (5′ to 3′) |
|---|---|---|
| ASB11 | qPCR | F CCTGCTAACCGACTATGGAGC |
| | | R TAGGAGGAATCGTTCGAGTGG |
| ATF6 | | F TCCTCGGTCAGTGGACTCTTA |
| | | R CTTGGGCTGAATTGAAGGTTTTG |
| BIK | | F GACCTGGACCCTATGGAGGAC |
| | | R CCTCAGTCTGGTCGTAGATGA |
| GAPDH | | F GTCTCCTCTGACTTCAACAGCG |
| | | R ACCACCCTGTTGCTGTAGCCAA |
| NFYB | | F ATGACAATGGATGGTGACAGTTC |
| | | R CTAGCCACGTTTGCTATTGGA |
| NFYC | | F GGAGGATTTGGTGGTACTAGCA |
| | | R GCACTCGGAAGTCTTTCACTG |
| p53 | | F ACTTGTCGCTCTTGAAGCTAC |
| | | R GATGCGGAGAATCTTTGGAACA |
| PERK | | F ACGATGAGACAGAGTTGCGAC |
| | | R ATCCAAGGCAGCAATTCTCCC |
| XBP1 | | F CCCTCCAGAACATCTCCCCAT |
| | | R ACATGACTGGGTCCAAGTTGT |
| XBR1s | | F AAGCACCTCTCAGCCCCTCA |
| | | R GCTTTGGGCAGTGGCTGGAT |
| XBP1 | PCR | F TTACGAGAGAAAACTCATGGC |
| (in- | | R GGGTCCAAGTTGTCCAGAATGC |
| tron) | | |
| A | ChIP- | F TTCTGGTAACAGGAGCACTGAG |
| (−63~ | qPCR | R GATCTCTTCTGCTATCCTAGCCGC |
| +165) | | |
| B | | F CTGTGGAAGCCAAGAAGTTTC |
| (−304~ | | R TGATTTGGCGACTGCATGTGC |
| −86) | | |
| C | | F CTCCAGCCTGGGCAACAGAG |
| (−870~ | | R TTGCTTGAACCTGGGAGGGG |
| −664) | | |
| D | | F CCTGCATGCTAGGAGAAATTCCCA |
| (−1623~ | | R AGGTCTCTGCCTTCATTCTTGGC |
| −1474) | | |
| Flag- | clon- | F AAAGAATTCACCATGCCGTGGGACGCGCGG |
| ANKRD9 | ing | R GCGCGTCGACGCCTTTGCCAGTGAGGTCCAAC |
| Flag- | | F AAAGAATTCACCATGGCGGAGGGCGGCAGC |
| ASB1 | | R GCGCGTCGACCTCATGGAGTAGAAACTTCT |
| Flag- | | F CCGGAATTCACCATGGATTTTACAGAGGCT |
| ASB3 | | R GCTTGTCGACTCCATCTTGAATAGCTGCCA |
| Flag- | | F ATTGAATTCACCATGGACGGCACCACTGCC |
| ASB4 | | R GCGCGTCGACATAAATAATTCCCTCTGGCTC |
| Flag- | | F CCGGAATTCACCATGTCGGTGTTAGAAGAA |
| ASB5 | | R GCGCGTCGACTCGATACTGTAAGAAATTCT |
| Flag- | | F ATTGAATTCACCATGCCGTTCCTGCACGGC |
| ASB6 | | R AATTGTCGACGATGTCATCTTCCACGGAGC C |
| Flag- | | F ATGTTACACCATCATTGTCGAAGGAACC |
| ASB7 | | R CGCGGATCCGATATCATCAAATTTGTGTTT |
| Flag- | | F CCGGAATTCACCATGAGTTCCAGTATGTGG |
| ASB8 | | R GCGCGTCGAC TTCTAAAAGT AACAGGTATT |
| Flag- | | F ATGGATGGCAAACAAGGGGGCATGGATG |
| ASB9 | | R CGCGGATCCAAGATGTAGGAGAAACTGTTTC |
| Flag- | | F GCGGAATTCACCATGCTCATGAGTTGGTCT |
| ASB10 | | R CCTTGTCGACGTAGAGCACGCCCTCAAAATC |
| Flag- | | F ATGGAAGATGGTCCTGTTTTCTATGGCT |
| ASB11 | | R CGCGGATCCTTGGTATAGGAGGAATCGTTCG |
| Flag- | | F CCGGAATTCACCATGAGAATAGTTCTCCAA |
| ASB12 | | R GCGCGTCGACCAGTTGGTGTTTTAGGTACG |
| Flag- | | F ATTGAATTCACCATGGAGCCCCGGGCGGCGGAC |
| ASB13 | | R GCGCGTCGACGTTGTAGGAGAGGTAATCAAT |
| Flag- | | F ATGGATAATTACACCAGCGATGAAGACA |
| ASB14 | | R TTGCGTCGACCCAGGTTCCTGTAAAAATTC |
| Flag- | | F ATGGATACTAATGATGACCCTGATGAAG |
| ASB15 | | R CGCGGATCCTGTCAATTTTAGCTCTTGTCC |
| Flag- | | F ACGGAATTCACCATGGCAAGAGAGACCTTC |
| ASB16 | | R AATTGTCGACCTGGATGCACCCCTCCACAC |
| Flag- | | F CCGGAATTCACCATGAGTAAATCTACTAAATTA |
| ASB17 | | R GCGCGTCGACGATTTCTAAATTCAGATAGTT |
| Flag- | | F TGAATTCACCATGTCCAACTCGGATTACCTT |
| ASB18 | | R CCATGTCGACGTGCAAAACACCCTGTGGCT |
| Flag- | | F AGAATTCACCATGTACCTAGAACACACCAG |
| CIS | | R AATTGTCGACGAGCTGGAAGGGGTACTGTC |
| Flag- | | F AGAATTCACCATGGCGGCGCCCGAGGCCTG |
| Muf1 | | R AATTGTCGACCATGGTGCTAACATAATCTG |
| Flag- | | F TTGGAATTCACCATGGGAGGAGCTGTGAGT |
| PCMTD1 | | R GCGCGTCGACTTTGTCTCTAAAATATGTCA |
| Flag- | | F AGAATTC ACCATGGGCGGTGCTGTGAGTGC |
| PCMTD2 | | R AATTGTCGACTTTTTCTCTGTAATAAAGCA |
| Flag- | | F AAAGAATTCACCATGACCGCCCCGGGCAGC |
| RAB40A | | R GCGCGTCGACAGAAATTTTGCAGCTGTTTCTGG |
| Flag- | | F AAAGAATTCACCATGAGCGCCCTGGGCAGC |
| RAB40B | | R GCGCGTCGACAGAAATTTTGCAGCTGTTTCTGG |
| Flag- | | F TTAGAATTCACCATGGGCTCGCAGGGCAGT |
| RAB40C | | R TTGCGTCGACGGAGATCTTGCAGTTACTCC |
| Flag- | | F AGAATTCACCATGGGTCAGAAGGTCACTGG |
| SSB1 | | R AATTGTCGACCTGGTAGAGGAGGTAGGCCT |
| Flag- | | F AGAATTCACCATGGGCCAGACAGCTCTGGC |
| SSB2 | | R AATTGTCGACCTGGTAGAGCAGGTAGCGCT |
| Flag- | | F AGAATTCACCATGGCCAGACGCCCCCGGAA |
| SSB3 | | R AATTGTCGACGGTCCGGCGGCAGCGCTTCC |
| Flag- | | F AGAATTCACCATGGGCCAGAAGCTCTCGGG |
| SSB4 | | R AATTGTCGACCTGGTACTGCAGATAGTTTT |
| Flag- | | F AGAATTCACCATGGTAGCACACAACCAGGT |
| SOCS1 | | R AATTGTCGACAATCTGGAAGGGGAAGGAGC |
| Flag- | | F AGAATTCACCATGACCCTGCGGTGCCTTGA |
| SOCS2 | | R AATTGTCGACTACCTGGAATTTATATTCTT |
| Flag- | | F AGAATTCACCATGGTCACCCACAGCAAGTT |
| SOCS3 | | R AATTGTCGACAAGCGGGGCATCGTACTGGT |
| Flag- | | F AGAATTCACCATGGCAGAAAATAATGAAAA |
| SOCS4 | | R AATTGTCGAC GCATTGCTGT TCTGGTGCAT |
| Flag- | | F AATATCGATACCATGGATAAAGTGGGAAA |
| SOCS5 | | R AATTGTCGACCTTTGCCTTGACTGGTTCTC |
| Flag- | | F AGAATTCACCATCAAGAAAATTAGTCTTAA |
| SOCS6 | | R AATTGTCGACGTGCTTCTCCTGTAAATAAT |
| Flag- | | F CCGGAATTCACCATGGTGTTCCGCAACGTGGGT |
| SOCS7 | | R GCGTGTCGACCGTGGAGGGTTCCACCTCTTG |
| Flag- | | F TGAATTCACCATGTATGCAGCAGTGGAACA |
| TULP4 | | R CCATGTCGACTTTGAGGCGCTGAGTCACGT |
| Flag- | | F AGAATTCACCATGGCCAGCTTTCCCCCGAG |
| WSB1 | | R AATTGTCGACAATACGATACGAGAGAAACT |
| Flag- | | F ATTGAATTCACCATGGAGGCCGGAGAGGAACCG |
| WSB2 | | R GCGCGTCGACAAAAGTCCTGTATGTGAGGAA |
| BIK | | F TGAATTCACCATGTCTGAAGTAAGACCCCT |
| | | R CCATGTCGACTCACTTGAGCAGCAGGTGCA |
| Flag- | | F AAAGAATTCACCATGGCGCACGCTGGGAGA |
| BCL2 | | R ATATGTCGACCTTGTGGCCCAGATAGGCA |
| 3xFlag- | | F GAAGATCTTCATGTCTGAAGTAAGACCCCT |
| BIK | | R GCTCTAGATCACTTGAGCAGCAGGTGCAG |
| Myc- | | F ATGGAAGATGGTCCTGTTTTCTATGGCT |
| ASB11 | | R CGCGGATCCTTGGTATAGGAGGAATCGTTCG |
| Myc- | | F ATGGAAGATGGTCCTGTTTTCTATGGCT |
| DNASB11 | | R GGAGGATCCAGGTGGGCCTTCACGGAGCAA |
| V5-ROC2 | | F TGAATTCACCATGGCCGACGTGGAAGACGG |
| | | R AATTGTCGACTTTGCCGATTCTTTGGACCA |
| Flag- | | F ATGGAAGATGGTCCTGTTTTCTATGGCT |
| ASB11 | | R CGCGGATCCTTGGTATAGGAGGAATCGTTCG |
| Flag- | | F ATGGAAGATGGTCCTGTTTTCTATGGCT |
| DNASB11 | | R GGAGGATCCAGGTGGGCCTTCACGGAGCAA |
| Myc- | | F ATAGGATCCACCATGGTGGTGGTGGCAGCC |
| XBP1(s) | | R ATATGTCGACGACACTAATCAGCTGGGGAA |
| BIK | | F TTCACCACACTTAGGGAGAACATAATG |
| (115R) | | R CATTATGTTC TCCCTAAGTG TGGTGAA |
| BIK | | F CTGCACCTGCTGCTCAGGTGAGTCGAC |
| (160R) | | R GTCGACTCACCTGAGCAGCAGGTGCAG |
| BIKDD | | F GTTCTTGGCATGGATGACGATGAAGAG |
| | | R CTCTTCATCGTCATCCATGCCAAGAAC |
| pLAS5W- | | F ATTGCTAGCACCATGGAAGATGGTCCTGTT |
| Myc- | | |
| ASB11 | | R GGAATTCTTATCACAGCAGGTCCTCCTCGC |
| ASB11 | | F GATGCTAGCATGTTATAGAACAATGTACATCAC |
| promoter | | |
| 1949 | | R ATCCTCGAGTTTGGCTTATGCTCTGTATAGGGT |
| ASB11 | | F GATGCTAGCACGCGCCCGGCCAGGTAAATGG |
| promoter | | |
| 478 | | R ATCCTCGAGTTTGGCTTATGCTCTGTATAGGGT |
| ASB11 | | F CTATTGGATGTTTCTGAAGGAAGTTTCTTT |
| promoter | | |
| 478 dNFY | | R AAAGAAACTTCCTTCAGAAACATCCAATAG |

RNA Interference

Lentivirus-based constructs were obtained from National RNAi Core Facility, Taiwan. The shRNA target sequences are listed in Supplementary Table 3. To generate recombinant lentivirus carrying shRNAs, 293FT cells were co-transfected with the packaging plasmid pCMVDR8.91, envelope plasmid pMD.G, and shRNA expressing construct. For infection, the viral stock was supplemented with 8 μg/ml polybrene and infected cells were selected by appropriated agents.

SUPPLEMENTARY TABLE 3
Targeting sequences for shRNAs

| | | Target sequence |
|---|---|---|
| Gene | Clone | (5′ to 3′) |
| Cul2 | #1 | GCAAGCTACATCGGATGTATA |
| Cul2 | #2 | CGTTTGCAGTTGATGTGTCTT |
| Cul5 | #1 | GCAGACTGAATTAGTAGAAAT |
| Cul5 | #2 | CGCTGTATTGTTTGCATGGAA |
| ASB11 | #1 | CAGTGCTGCATGTGTCAATGT |
| ASB11 | #2 | GGATAGCAGAAGAGATCTATG |
| ATF6 | #1 | CCTAGTCCAAAGCGAAGAGTT |
| ATF6 | #2 | AGAACTGTCTCGTACTAGAAT |
| BIK | #1 | TCTTGATGGAGACCCTCCTGT |
| NFYB | #1 | CACAACATCATATCAACAGAT |
| NFYB | #2 | CCAAAGAATGTGTTCAAGAAT |
| NFYC | #1 | CTGGCTCGTATTAAGAAGATT |
| NFYC | #2 | ATGATATCGCCATGGCAATTA |
| PERK | #1 | CCCAAACTGATTATAGGTAAC |
| PERK | #2 | GAAACAGCTATTCTCATAAAG |
| XBP1 | #1 | GCCTGTCTGTACTTCATTCAA |
| XBP1 | #2 | GAACAGCAAGTGGTAGATTTA |

Immunoprecipitation and Western Blot

Cell extraction was performed with RIPA lysis buffer containing 50 mM Tris (pH 8.0), 0.15 M NaCl, 1% NP40, 1% sodium deoxycholate, 0.1% SDS, 1 mM PMSF, 1 pg/ml aprotinin, and 1 pg/ml leupeptin. Western blot was performed with the standard protocol. For an efficient detection of BIK by Western blot, Tricine-SDS-PAGE was used. Western blot analyses of other proteins were performed with Glycine-SDS-PAGE. Immunoprecipitation was performed and analyzed as previously described. Briefly, cell lysates were incubated with primary antibody for overnight. The Pure Proteome Protein A/G Magnetic Beads (LSKMAGA/G10, EMD Millipore) were then added into cell lysates and incubated for 1.5 h. The beads were washed with RIPA lysis buffer and the bound proteins were analyzed by Western blot.

Ubiquitination Assays

For in vitro ubiquitination assay, ASB11-based Cul5 E3 ligase complex and 3× Flag-BIK were separately purified using anti-Flag M2 affinity agarose gel (Sigma) from lysates of 293T cells transfected with 3× Flag-BIK or co-transfected with Flag-ASB11, Myc-Cul5, V5-Roc2, T7-ElonginC, and HA-ElonginB. The E3 ligase complex bound on beads was incubated in a 20 μl ubiquitination reaction mixture containing 40 ng yeast E1 enzyme, 500 ng E2 enzyme (UbcH5a), 300 ng 3× Flag-BIK and other components as described previously (Yuan, W. C. et al. A Cullin3-KLHL20 Ubiquitin ligase-dependent pathway targets PML to potentiate HIF-1 signaling and prostate cancer progression. Cancer Cell 20, 214-228 (2011)). The E1, E2, His-ubiquitin and other related reagents used in this assay were purchased from R&D Systems.

For in vivo ubiquitination assay, cells were transfected with various constructs and His-ubiquitin and treated with 1 μM MG132 for 16 h. Cells were lysed under denaturing conditions by buffer A (6 M guanidine-HCl, 0.1 M Na2HPO4/NaH2PO4, pH 8.0, and 10 mM imidazole), and lysates were incubated with Ni-NTA Sepharose for 2 h at 4° C. The beads were washed once with buffer A, twice with buffer A/TI (1 vol buffer A: 3 vol buffer TI [25 mM Tris-HCl, pH 6.8, and 20 mM imidazole]), and three times with buffer TI, and then incubated in the sample buffer at 95 C for 5 min. In all experiments, equal expression of His-ubiquitin was verified by Western blot analysis.

Apoptosis Assay

Apoptosis was analyzed as previously described. Briefly, cells were seeded at a density of 1×10⁶ cells in 6-cm dish overnight. The cells were then incubated with doxorubicin or tunicamycin for various time points. Cells were harvested and DNA fragmentation was measured by Cell Death ELISA Kit (Roche).

mRNA Extraction and RT-qPCR

Total mRNA was extracted from cells using TRIZOL reagent (Invitrogen), and equal amounts of RNA were reverse transcribed to cDNA using the iScript™ cDNA Synthesis Kit (Bio-Rad). Quantitative real-time PCR was performed using the Power SYBR Green PCR Master kit (Applied Biosystems). Amplification was performed on an ABI 7500 Fast Real-Time PCR system and GAPDH was used as an internal control.

Luciferase Reporter Assay

Cells were co-transfected with pGL3-based reporter construct, pTK-renilla plasmid, together with other constructs for 24 h. Luciferase reporter assay was performed by the dual-luciferase reporter assay system (Promega) according to the manufacturer's instructions. The relative promoter activity was expressed as the fold change in firefly luciferase activity after normalization to the renilla luciferase activity.

ChIP Assay

ChIP assay was performed as previously described. Brief, 293T cells were seeded at a density of 1×10⁷ cells in 10-cm dish overnight. The cells were treated with tunicamycin for 4 h, and then fixed with 1% formaldehyde. ChIP was proceeded with XBP1 antibody or ChIP grade rabbit IgG (as a control). Enrichment of promoter binding level was analyzed by qPCR. The qPCR primers for ChIP assay are listed in Supplementary Table 2.

MTT Assay

MDA-MB157 and MDA-MB 468 cells were seeded at a density of 5×10³ cells and Hs578T cells were at 2×10³ cells in 96-well plates. Cells were transfected with BIKDD. In the next day, cells were treated with IRE1α inhibitor or DMSO for 48 h and then with 0.4 mg/ml methylthiazolyldiphenyltetrazolium bromide (MTT) (Sigma) for 2 h. Cells were dissolved in DMSO, followed by absorbance measurement at 590 nm. The combination index (CI) was calculated using the equation: CI=C_{A}/IC50_{A}+C_{B}/IC50_{B}. C_{A} and C_{B} represent the concentrations of the two agents for combined treatment. IC50_{A} and IC50_{B} are the IC50 values for each single treatment, which were determined by treating cells with various dosages of each agent followed by MTT assay. (CI<1) indicates a synergistic effect, (CI>1) corresponds to an antagonistic effect, and (CI=1) represents an additive effect.

Animal Experiments

All mice were maintained according to the guidelines of animal ethical regulations, and all animal studies were approved by the Experimental Animal Committee, Academia Sinica. Five-week-old female BALB/cAnN.Cg-Foxnlnu/CrlNarl nude mice (National Laboratory Animal Center, Taipei, Taiwan) were inoculated in the mammary fat pad with 2×10⁶ Hs578T cells transiently expressing BIKDD or control vector. Seven days later, DMSO or STF-083010 (40 mg/kg) was intraperitoneally administrated every 3 days. For BIKDD gene therapy model, five-week-old female BALB/cAnN.Cg-Foxnlnu/CrlNarl nude mice (National Laboratory Animal Center, Taipei, Taiwan) were inoculated in the mammary fat pad with 2×10⁶ Hs578T cells. Twenty-eight days later, DMSO or STF-083010 (40 mg/kg) was intraperitoneally administrated every 3-4 days. Control vector or VISA-BIKDD (0.75 mg/kg) was first incubated with the in vivo-jetPEI delivery reagent (PEI; Polyplus Transfection, New York, USA) for 15 min and then the complex was intratumorally injected every 7 days starting at day 28. For both models, tumors were measured every 3 or 4 days, and their volumes were calculated using the equation: mm³=n/6×length(mm)×(width(mm))².

Statistical Analysis

Statistical analysis was performed using two-tailed Student's t tests for comparisons between two groups and one-way or two-way analysis of variance (ANOVA) with Tukey's post test for multigroup comparisons. All statistical analyses were conducted at a significance level of p<0.05.

### Example 1 Identification of CRL5^{ASB11} as a BIK Ubiquitin Ligase

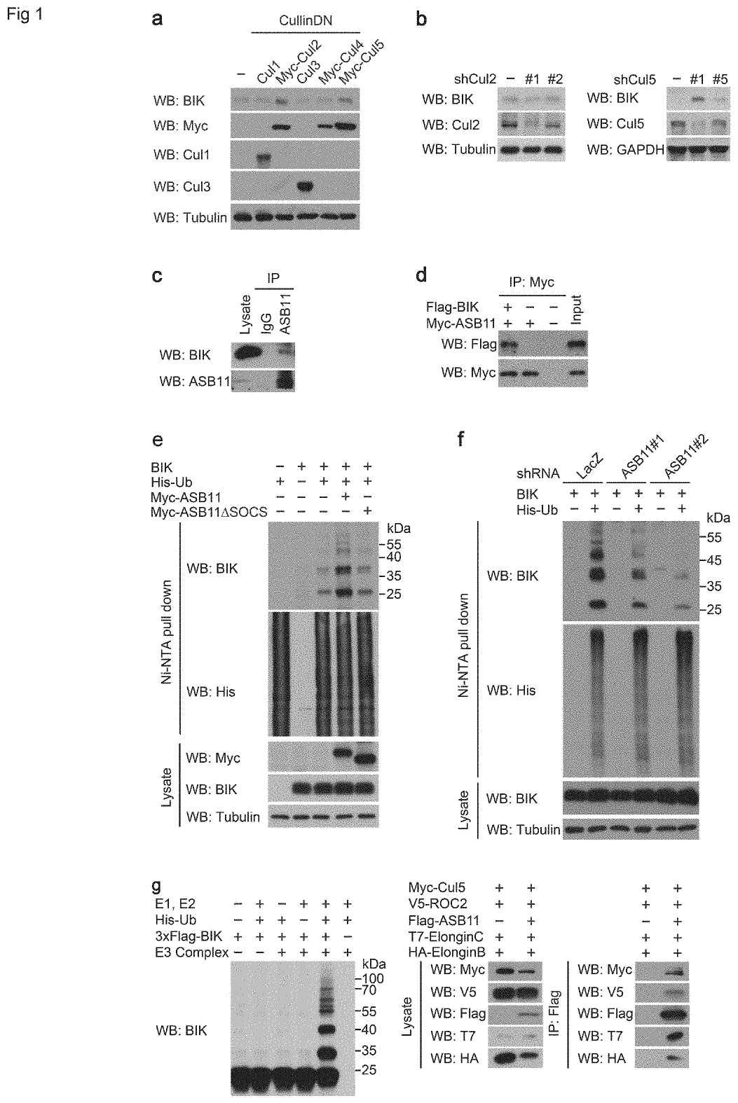

To identify ubiquitin ligase for BIK, we focused on Cullin-RING ubiquitin ligases (CRLs), which comprise the largest ubiquitin ligase family (Petroski, M. D. & Deshaies, R. J. Function and regulation of cullin-RING ubiquitin ligases. Nature reviews 6, 9-20 (2005)). By using dominant-negative (DN) mutant of each Cullin, we found that Cul2 and Cul5 mutants elevated the expression of BIK protein but not BIK mRNA (FIG. 1a). Since DN mutants of Cul2 and Cul5 are promiscuous due to the sharing of ElonginB/C subunits by the two CRL complexes, we further validated the role of Cul2 and Cul5 using knockdown approach. Remarkably, BIK expression was upregulated by two independent Cul5 shRNAs and this effect correlated with knockdown efficiency (FIG. 1b). However, Cul2 knockdown could not elevate BIK expression. These findings indicate an effect of CRL5 on BIK regulation. CRL5 complex contains ROC2, Cul5, ElonginB, ElonginC and one of many substrate adaptors with a SOCS box (Lydeard, J. R., Schulman, B. A. & Harper, J. W. Building and remodelling Cullin-RING E3 ubiquitin ligases. EMBO Rep 14, 1050-1061 (2013)). To identify the Cul5 substrate adaptor responsible for BIK regulation, we expressed each of the 39 Cul5 substrate adaptors and tested their interaction with endogenous BIK by immunoprecipitation. This analysis identified ANKRD9, ASB11, and ASB17 as BIK-interacting proteins. Among them, only ASB11 could potentiate BIK ubiquitination when expressed in vivo. Similar to BIK, ASB11 is an ER-residing protein. Immunoprecipitation analysis demonstrated the interaction of endogenous ASB11 with endogenous BIK in vivo and a direct interaction between purified ASB11 and BIK in vitro (FIG. 1c, d). Furthermore, ASB11 knockdown impaired the in vivo interaction of BIK with Cul5. These findings are consistent with the adaptor role of ASB11 in recruiting BIK to the CRL5 complex. Similar to other Cul5 substrate adaptors, ASB11 possesses a SOCS box for binding Cul5 and ElonginB/C. We found that deletion of SOCS box abolished the capability of ASB11 to promote BIK ubiquitination (FIG. 1e). In addition, depletion of ASB11 in both 293T cells and H1299 cells reduced BIK ubiquitination level (FIG. 1f). In the in vitro ubiquitination assay, BIK ubiquitination was readily detected in the reaction supplemented with a full CRL5^{ASB11} complex, including ROC2, Cul5, ElonginB, ElonginC, and ASB11 (FIG. 1g). These results indicate that CRL5^{ASB11} is a direct and physiologically relevant ubiquitin ligase for BIK.

### Example 2 ASB11 Promotes BIK Proteasomal Degradation

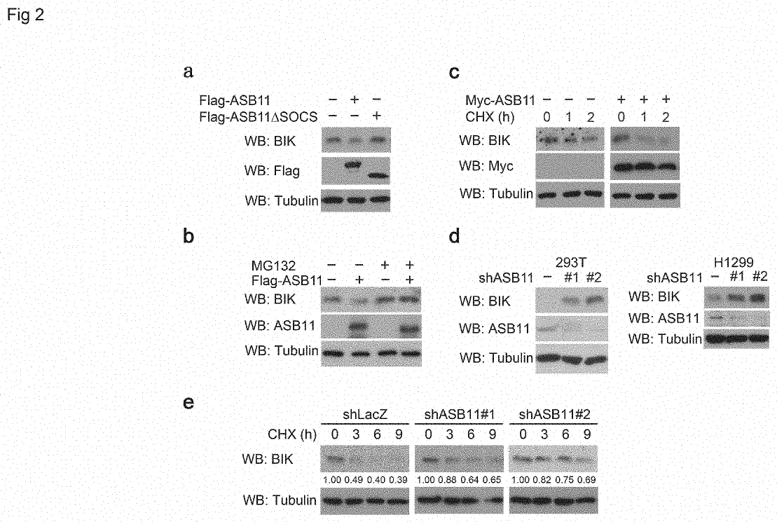

Next, we determined the consequence of BIK ubiquitination by CRL5^{ASB11}. Overexpression of ASB11, but not its SOCS box deletion mutant, decreased BIK protein level (FIG. 2a). This effect of ASB11 is reversed by treatment of cells with proteasome inhibitor MG132 (FIG. 2b). Using cycloheximide-chase assay, we found that ASB11 overexpression increased BIK protein degradation (FIG. 2c). In the reciprocal set of experiments, ASB11 knockdown in both 293T and H1299 cells elevated BIK level (FIG. 2d). Furthermore, ASB11 depletion increased BIK protein stability (FIG. 2e). These findings indicate that ASB11-mediated BIK ubiquitination promotes its proteasomal degradation.

### Example 3 ASB11 is a Transcriptional Target of XBPls

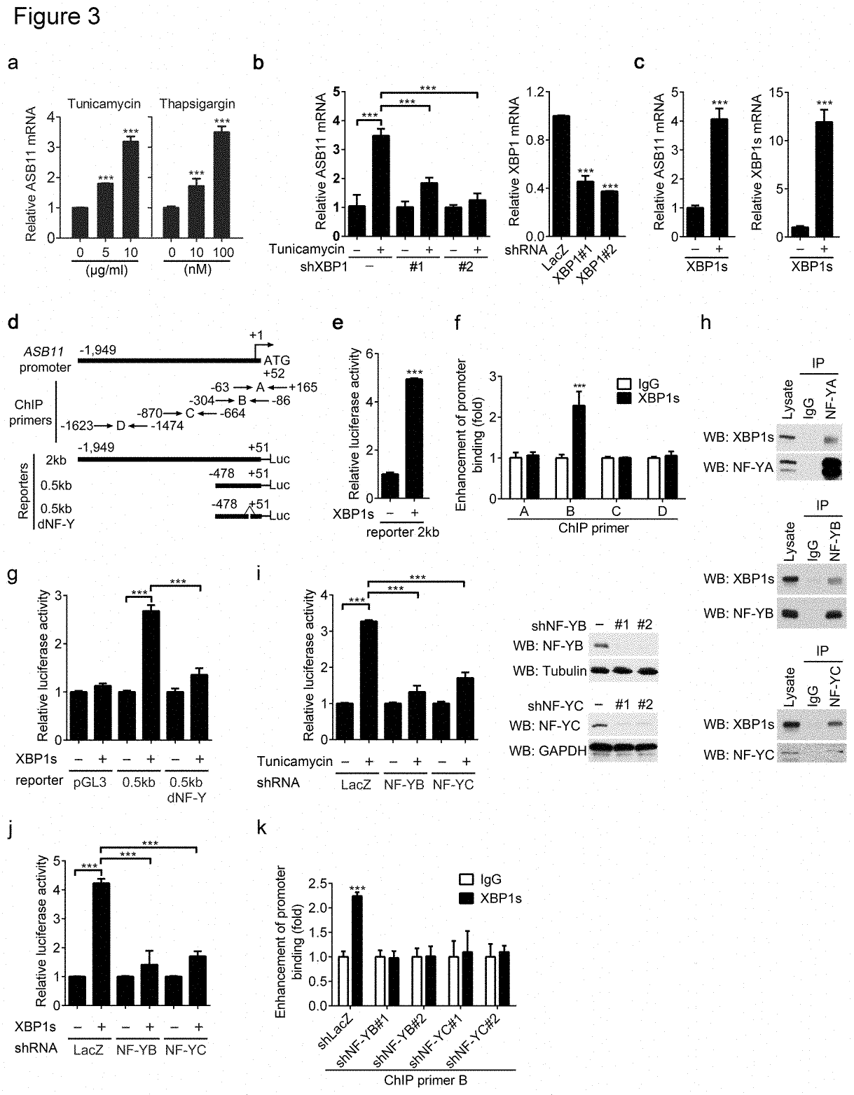

BH3-only proteins are usually regulated by various cellular stress signals. We investigated whether ASB11-mediated BIK ubiquitination could be regulated under cellular stress conditions. Remarkably, tunicamycin, which inhibits glycosylation to cause ER stress, upregulated the expression of ASB11 mRNA in multiple cell systems, including 293T, MDA-MB157, and MDA-MB468 cells (FIG. 3a). A similar upregulation of ASB11 mRNA was observed by another ER stressor, the calcium pump inhibitor thapsigargin (FIG. 3a). To dissect the molecular mechanism through which ER stress induces ASB11, we determined which of the three UPR branches is responsible for this effect. Blockage of IRE1/XBP1 axis by XBP1 depletion greatly suppressed tunicamycin-induced ASB11 mRNA expression, whereas ATF6 or PERK knockdown showed an opposite effect (FIG. 3b). In response to ER stress, XBP1 mRNA undergoes an IRE1a-dependent unconventional splicing to generate XPB1s, whose protein product functions as a transcription factor. Therefore, we set out to test whether ASB11 is a transcriptional target of XBPls. Overexpression of XBPls greatly increased ASB11 mRNA level and the activity of a luciferase reporter driven by a 2 kb-segment of the 5′ regulatory region of ASB11 gene (FIG. 3c-e). Chromatin immunoprecipitation (ChIP) analysis with four pairs of primers revealed that endogenous XBPls in tunicamycin-treated cells was specifically recruited to a region (−86 to −304) near the transcriptional start site of ASB11 gene (FIG. 3f). However, no authentic XBPls binding motif, such as UPRE, ERSE, ERSE-II, and AGCT core, could be found in this region. Instead, we identified an NF-Y binding motif in the position of −148 to −155. Since a previous report implicated the action of XBP1 on the NF-Y binding motif, we tested its importance. Accordingly, deletion of this motif abrogated XBP1s-induced ASB11 promoter activity (FIG. 3g), suggesting a cooperative function of these two transcription factors in ASB11 transcription. In line with this notion, immunoprecipitation analysis demonstrated the interaction of endogenous XBP1s with each of the NF-Y complex components, NF-YA, NF-YB, and NF-YC (FIG. 3h). Furthermore, depletion of NF-YB or NF-YC not only abrogated ER stress- or XBP1x-induced ASB11 promoter activity, but completely blocked the binding of XBPls to the ASB11 promoter in tunicamycin-treated cells (FIG. 3i-k). Thus, XBPls is recruited to the ASB11 promoter via NF-Y and this recruitment is crucial for the activation of ASB11 transcription under ER stress.

### Example 4 ER Stress Promotes BIK Degradation Through the Actions of ASB11 and p97/VCP

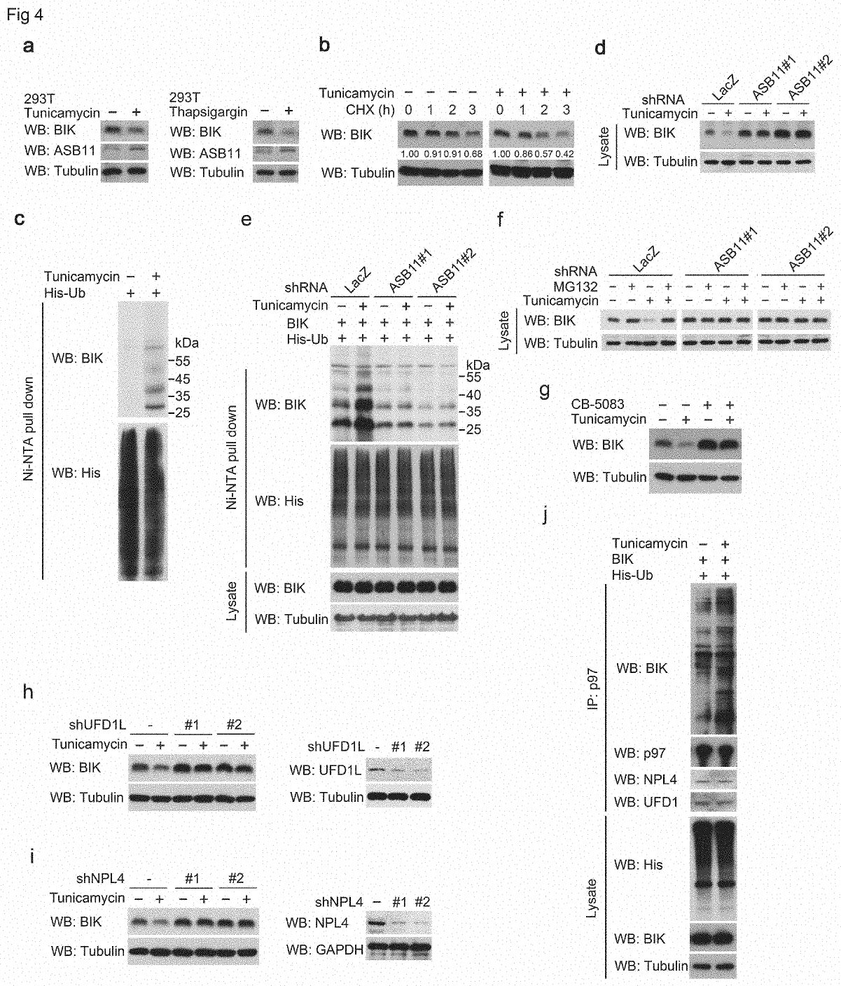

Consistent with the elevated ASB11 transcription under ER stress, tunicamycin and thapsigargin also elevated ASB11 protein expression and reduced BIK protein level in multiple cell lines (FIG. 4a). Tunicamycin also increased the ubiquitination and turnover of endogenous BIK (FIG. 4b, c). To corroborate that the reduced BIK level in ER stressed conditions is resulted from ASB11 upregulation, we examined ASB11 knockdown cells. Indeed, ASB11 knockdown in multiple cell lines, including 293T, H1299, and MDA-MB157 cells, all abrogated tunicamycin-induced BIK downregulation (FIG. 4d). Furthermore, tunicamycin stimulated BIK ubiquitination and proteasomal degradation were all reversed by ASB11 knockdown (FIG. 4e-f). In line with the critical role of XBP1 in ASB11 induction during ER stress, XBP1 knockdown also blocked tunicamycin-induced BIK downregulation and BIK ubiquitination. Our study thus identifies a role of ER stress in promoting BIK ubiquitination and degradation through XBP1-induced ASB11 upregulation.

BIK is an ER-residing transmembrane protein. We anticipated that extraction of ubiquitinated BIK from ER membrane would be critical for the subsequent degradation in proteasome. The AAA+ATPase p97, also known as valosin-containing protein, is responsible for segregating numerous ubiquitinated proteins from organelle membrane to facilitate their proteasomal degradation. Moreover, p97, together with its cofactor Ufd1-Np14 heterodimer, plays a crucial role in the degradation of ER residing proteins under ER stress, a process called ER-associated degradation (ERAD). We thus determined the function of p97-Ufd1-Np14 complex in ER stress-induced BIK degradation. Remarkably, administration of p97 inhibitor CB-5083 abrogated BIK downregulation induced by ER stress or ASB11 overexpression (FIG. 4g). Depletion of Ufd1 or Np14 also prevented ER stress-induced BIK degradation (FIG. 4h, i). Furthermore, immunoprecipitation analysis revealed an interaction of p97 with ubiquitinated BIK and this interaction was increased by tunicamycin treatment (FIG. 4k). These findings indicate a role of p97-Ufd1-Np14 complex in governing proteasomal degradation of ubiquitinated BIK under ER stressed conditions.

### Example 5 DNA Damage Induces p53-Dependent BIK Stabilization by Suppressing XBP1/ASB11 Axis

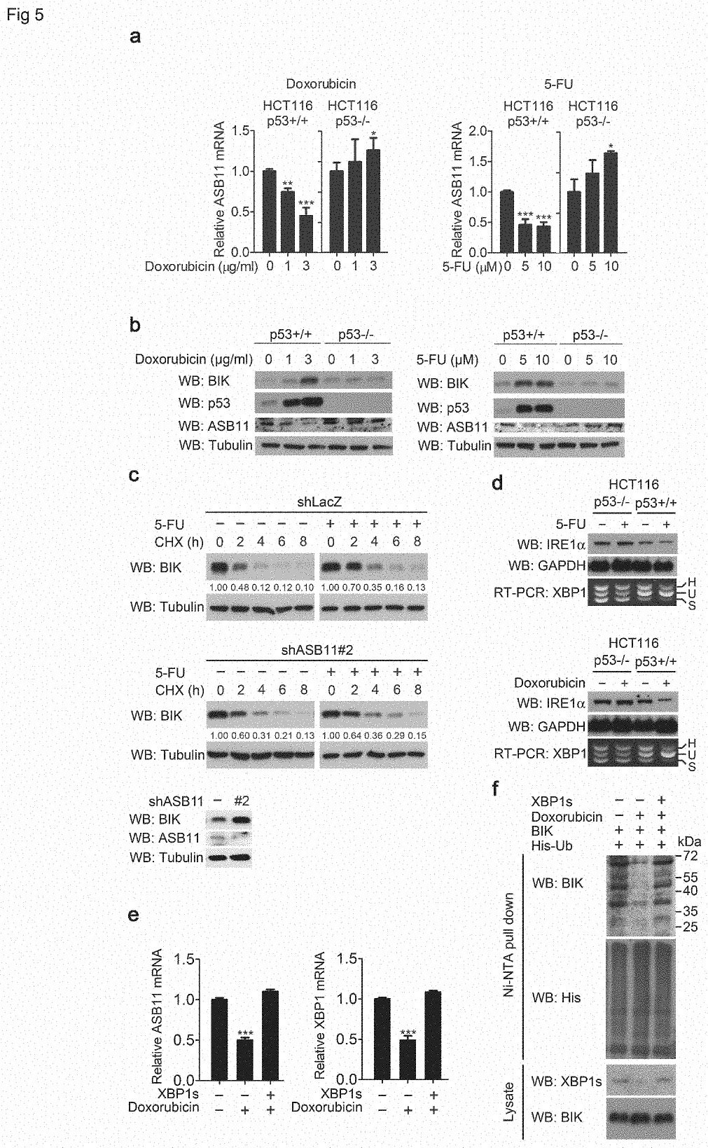

In a sharp contrast to ER stressed conditions, genotoxic agents such as doxorubicin and 5-fluorouracil (5-FU) reduced ASB11 mRNA level in p53-proficient HCT116 cells but not its p53-deficient counterpart (FIG. 5a). Similarly, doxorubicin downregulated ASB11 mRNA in p53-transfected H1299 cells but not in the parental, p53-deficient H1299 cells. In line with these findings, doxorubicin and 5-FU downregulated ASB11 protein and upregulated BIK protein levels in a p53-dependent manner (FIG. 5b). To determine whether protein stabilization attributes to the increased BIK level under DNA damaged conditions, we evaluated BIK protein degradation using cycloheximide-chase analysis. In p53-proficient HCT116 cells, BIK protein stability was increased upon 5-FU treatment (FIG. 5c, top panel). However, in ASB11-depleted HCT116 cells, BIK was readily stabilized and DNA damage hardly induced a further increase in BIK stability (FIG. 5c, second panel). These findings surprisingly discovered a DNA damage-induced and p53-dependent ASB11 downregulation, leading to BIK stabilization.

Next, we sought to unravel the underlying mechanism by which DNA damage reduces ASB11 expression. Since this effect is p53 dependent, we tested whether p53 regulates ASB11 transcription. Consistent with this idea, ASB11 mRNA expression was lower in p53-proficient HCT116 cells than its p53-deficient counterpart. Furthermore, p53 overexpression in H1299 cells decreased ASB11 mRNA level and promoter activity (Supplementary FIG. 4c, d). Nevertheless, we reasoned that p53 may not act on ASB11 promoter directly, as a previous meta-analysis indicated that the transcriptional repression effect of p53 is mainly mediated by indirect mechanisms. Notably, p53 was reported to repress IRE1/XBP1 pathway even in unstressed conditions. Consistently, we showed that doxorubicin and 5-FU reduced IRE1a level and XBP1 mRNA splicing through a p53-dependent manner (FIG. 5d). To provide a causal link of p53-dependent IRE1/XBP1 downregulation to ASB11 downregulation, we rescued XBP1s level by overexpression. Indeed, XBP1s overexpression restored ASB11 mRNA expression and BIK ubiquitination in doxorubicin-treated cells (FIG. 5e, f). Together, these data indicate that DNA damage-induced p53 represses the IRE1/XBP1 axis, leading to ASB11 downregulation and BIK stabilization.

### Example 6 Opposite Regulations of ASB11-Dependent BIK Ubiquitination by ER Stress and DNA Damage Govern the Life-Death Cell Fate

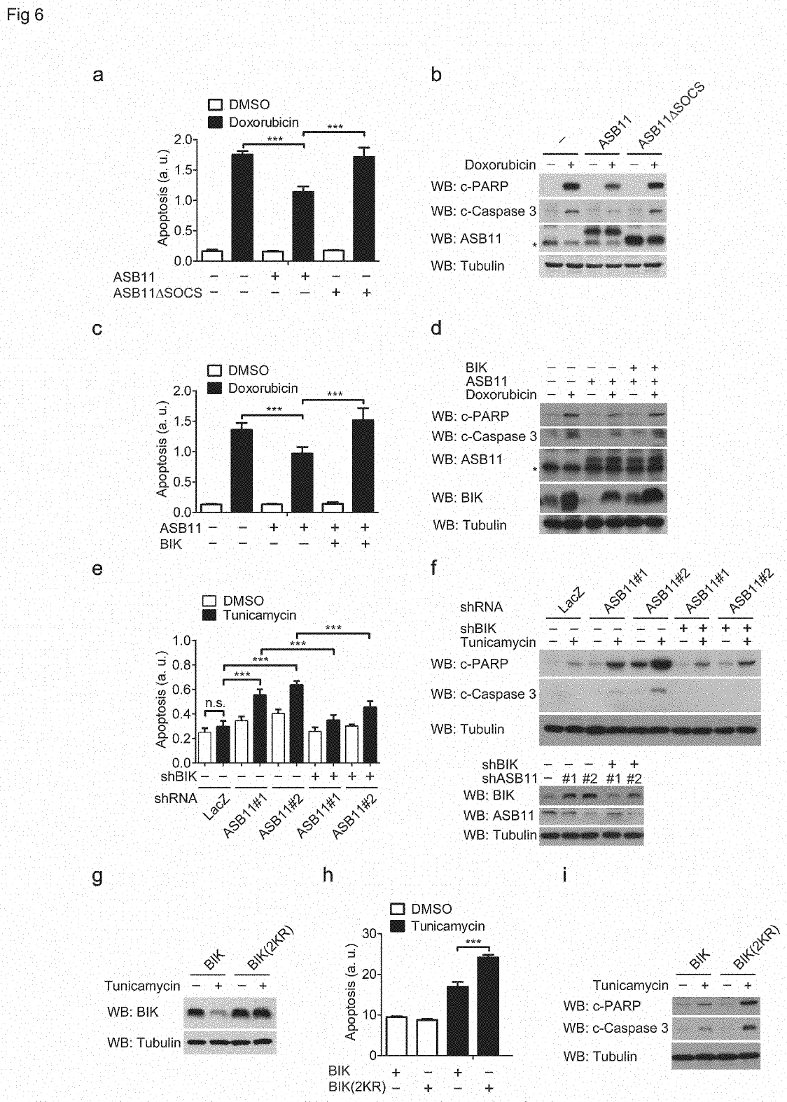

Having demonstrated that ER stress and DNA damage oppositely regulate ASB11 transcription and BIK protein stability, we next determined the impact of these BIK regulations on cell life-death decision. First, we interrogated the impact of DNA damage-induced ASB11 downregulation and BIK stabilization on cell apoptosis by enforced expression of ASB11. Importantly, expression of ASB11, but not its SOCS box deletion mutant, attenuated apoptotic death in doxorubicin-treated cells (FIG. 6a). ASB11 overexpression also diminished DNA damage-induced active caspase 3 and the cleaved form of PARP (FIG. 6b). To substantiate that these anti-apoptotic effects of ASB11 are mediated by BIK degradation, we rescued BIK expression in ASB11 overexpressing cells. Indeed, BIK overexpression completely reversed the inhibitory effects of ASB11 overexpression on DNA damage-induced cell apoptosis, caspase 3 activation, and PARP cleavage (FIG. 6c, d). These data indicate that DNA damage-induced ASB11 downregulation and BIK stabilization contribute in part to the apoptotic paradigm of DNA damage responses.

In contrast to DNA damage, ER stress upregulates ASB11 to induce BIK destabilization. We therefore investigated whether this regulation of ASB11/BIK axis could play a protective role against apoptosis. Indeed, at an early time point after receiving tunicamycin, while control cells did not show sign of apoptosis induction, ASB11-depleted cells exhibited a significant elevation in cell apoptosis, caspase 3 activation and PARP cleavage. Importantly, these effects of ASB11 knockdown were all reversed by BIK knockdown (FIG. 6e, f). These findings suggest that ER stress-induced and ASB11-mediated BIK degradation plays a pro-survival role to contribute to the adaptive phase of UPR. To further corroborate the effect of ASB11-dependent BIK ubiquitination on cell life-death fate under ER stress, we sought to generate an ASB11-resistant mutant of BIK. BIK contains two Lys residues (i.e., Lys115 and Lys160). Replacement of both residues with Arg (2KR mutant) completely abolished ASB11-dependent BIK ubiquitination and degradation. We thus expressed wild type BIK and BIK 2KR mutant in 293T cells. Of note, we chose the appropriate doses of two constructs so that the wild type and mutant BIK proteins were expressed at comparable levels in unstressed conditions (FIG. 6g). In contrast to wild type BIK, the expression of BIK(2KR) was not affected by tunicamycin, consistent with the ASB11-resistant property of this protein. Consequently, tunicamycin-induced caspase 3 activation, PARP cleavage, and cell apoptosis were all enhanced in cells expressing BIK(2KR), comparing to cells expressing wild type BIK (FIG. 6h, i). These findings indicate that ASB11-induced BIK destabilization protects cells from ER stress-induced apoptosis.

### Example 7 Targeting ASB11-Dependent BIK Degradation Pathway Enhances the Anti-Tumor Effect of BIKDD

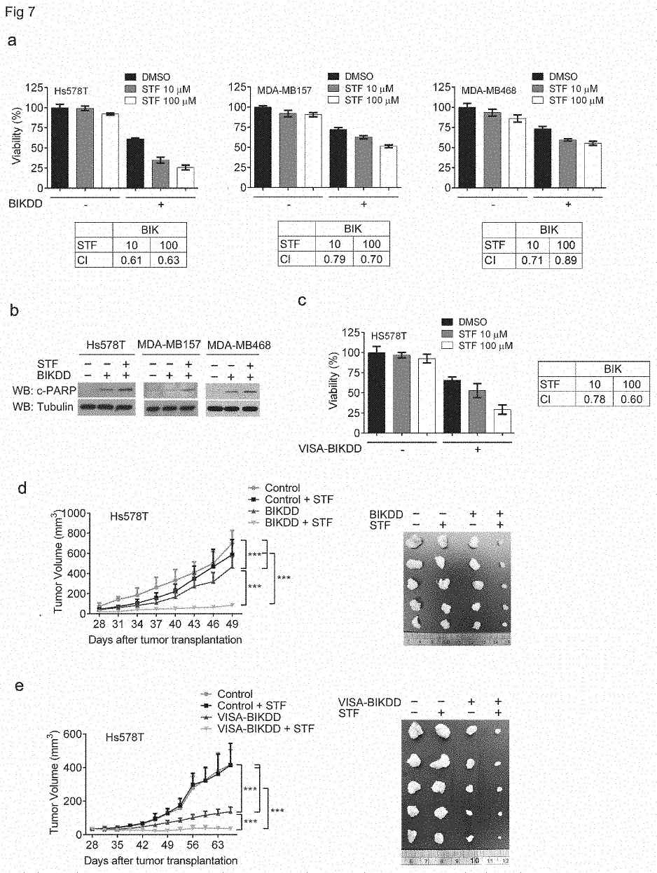

BIKDD, in which the Thr33 and Ser35 residues are replaced with Asp residues to mimic its phosphorylation form, has a higher affinity to the pro-survival Bcl-2 family proteins and thus represents an active mutant of BIK. Due to its potent pro-apoptotic activity, tumor-selective expression of BIKDD has been demonstrated as an effective anti-tumor strategy in several preclinical models and can even eliminate tumor-initiating cells. However, the labile feature of BIKDD has been a limitation. Since BIKDD also underwent ASB11-dependent and tunicamycin-induced ubiquitination and degradation, we reasoned that targeting this BIKDD degradation pathway would enhance its stability and anti-tumor efficacy. One way to inhibit ASB11-dependent BIKDD degradation is the administration of IRE1α inhibitor. As to the cancer type, we focused on triple negative breast cancer (TNBC) for two reasons. First, TNBC is a highly aggressive disease with limited treatment options. Second, BIKDD is particularly prone to degradation in TNBC due to the frequent upregulation of IRE/XBP1 axis, thus making its stabilization an important issue. We showed that a representative compound of the invention, the IRE1μ inhibitor STF-083010, elevated BIKDD levels in multiple TNBC cell lines, including Hs578T, MDA-MB157 and MDA-MB468. Consequently, STF-083010 synergized with BIKDD in the killing of these TNBC cells (FIG. 7a). The increased apoptotic effect of combinatory treatment was further evidenced by the increased level of cleaved PARP (FIG. 7b). The killing effect of BIKDD on TNBC cells was also enhanced by combined administration of another IRE1α inhibitor 4μ8C. Besides the utilization of CMV-BIKDD, we tested the synergistic combinatory effect of IRE1α inhibitor together with VISA-BIKDD, which allows a selective expression of BIKDD in breast cancer cells. Importantly, synergistic killing effect was observed by co-administration of VISA-BIKDD and STF-083010 (FIG. 7c). Thus, our data indicates a beneficial effect of combinatory application of BIKDD and IRE1α inhibitor for treating TNBC.

Next, we evaluated the anti-tumor activity of such combinatory treatment strategy using in vivo models. To this end, Hs578T cells expressing BIKDD or vector control were orthotopically transplanted to the mammary fat pad of nude mice, followed by administration of STF-083010 (see Materials and Methods for details). While administration of BIKDD or STF-083010 alone resulted in a modest reduction of tumor growth, combined treatment greatly suppressed tumor growth (FIG. 7d). To improve the therapeutic feasibility, we adopted a previously established gene therapy protocol via liposome-assisted delivery of VISA-BIKDD³⁶ and combined this gene therapy approach with STF-083010 administration for treating nude mice bearing orthotopic breast tumors derived from Hs578T cells. With this model, we again observed a significant enhancement of anti-tumor effect by combined administration of BIKDD and STF-083010, comparing to treatment with BIKDD alone (FIG. 7e). Importantly, the body weights of mice were not altered in either model, implicating the lack of toxicity. These findings indicate that compounds and compositions of this disclosure that target the ASB11-dependent BIK degradation pathway synergistically enhance the anti-tumor efficacy of BIKDD-based gene therapy.

### Example 8. Preparation of BIKDD-Comprising Nanoparticles

In one embodiment, the nanoparticles described herein are prepared by a process described below.

First, DOTAP (1,2-dioleoyl-3-trimethylammonium-propane) is removed from a 4°, warmed to room temperature using a water bath set to 60° C. Second, 99 mg DOTAP is placed in 250 mL round bottom flask. Third, 14 mL chloroform and 14 mL of methanol are added to the round bottom flask. Fourth, the flasks are agitated for 5 mins to mix the contents thoroughly. Fifth, the organic solvent is removed by rotary evaporation with a water bath set to 60° C. Sixth, the flask is placed in a vacuum desiccator overnight at room temperature, protected from light. Seventh, add 8.8 mL double distilled water to the flask and allow to dissolve for 15 min at room temperature. Eight, in an ultrasonic cleaner water bath (200 W) in a round bottom flask, sonicate for 5 min, and then using an ultrasonic cell disrupter (200 W) 5 min ultrasound (ultrasonic 5s, intermittent 5s). Ninth, prepare liposomes by sequentially filtering through 0.45 micoron, then 0.22 micron membrane filters. Tenth, the liposomes are then stored in 4° C. protected from light under inert gas.

Next, C-VISA_BIKDD liposomes are prepared by diluting 1 microliter C-VISA-BIKDD vector (which contains 60 micrograms of the vector) into 60 microliters of the liposome solution and 40 microliters of 1×PBS solution, using gently pipette tip mixing to yield a concentration of 100 microliters of 0.6 micrograms C-VISA-BIKDD plasmid solution/microliter. The size was measured by dynamic light scattering detection to confirm that the nanoparticle size after plasmid incorporation ranges from 150 nm-500 nm.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method for preventing and/or treating a subject having cancer, comprising administering to said subject a composition comprising an active BIK (BIKDD) and a BIKDD ubiquitination pathway inhibitor or blocker.

2. The method of claim 1, wherein the BIKDD ubiquitination pathway is the BIK degradation pathway.

3. The method of claim 1, wherein in the pathway, CRL5^{ASB11} is used as an ubiquitin ligase targeting BIK for ubiquitination and proteasomal degradation.

4. The method of claim 1, wherein the depletion of ASB11 reduces BIK ubiquitination level resulting in the inhibition or blockage of the BIKDD ubiquitination pathway.

5. The method of claim 1, wherein the overexpression of ASB11 decreases BIK protein level and increases BIK protein degradation.

6. The method of claim 5, wherein ASB11 is transcriptionally activated by XBP1.

7. The method of claim 1, wherein in the pathway, expression of ASB11 is upregulated by tunicamycin or a calcium pump inhibitor.

8. The method of claim 1, wherein ER stress promotes BIK ubiquitination and degradation through XBP1-induced ASB11 upregulation.

9. The method of claim 1, wherein a genotoxic agent acts through p53 to down-regulate IRE1α and ASB11, thereby stabilizing BIK.

10. A method for preventing and/or treating a cancer in a subject, comprising administering to said subject a combination of a BIK gene therapy and an IRE1α inhibitor.

11. The method of claim 10, wherein the BIK gene therapy and the administration of the IRE1-alpha inhibitor are performed separately or simultaneously.

12. The method of claim 10, wherein the BIK gene therapy comprises the administration of a BIKDD lipid nanoparticle.

13. The method of claim 12, wherein the BIKDD lipid nanoparticle comprises or is selected from: BIKDD, C-VISA BIKDD: liposome, CMV-BIKDD, or SV-BIKDD.

14. The method of claim 10, wherein the IRE1α inhibitor is 7-Hydroxy-4-methyl-2-oxo-2H-1-benzopyran-8-carboxaldehyde 4μ8C (4-methyl umbelliferone 8-carbaldehyde), IRE1 Inhibitor I (N-[(2-Hydroxy-1-naphthalenyl)methylene]-2-thiophenesulfonamide; STF-083010), 3-ethoxy-5,6-dibromosalicylaldehyde, (R)-2-(3,4-dichlorobenzyl)-N-(4-methylbenzyl)-2,7-diazaspiro[4.5]decane-7-carboxamide (GS K2850163), 3,6-DMAD hydrochloride, Toyocamycin (Vengicide), KIRA6, APY29, Kira8 (AMG-18), MKC9989, or MKC8866.

15. The method of claim 1, wherein the cancer is selected from: neuroblastoma; lung cancer; bile duct cancer; non-small cell lung carcinoma; hepatocellular carcinoma; head and neck squamous cell carcinoma; squamous cell cervical carcinoma; lymphoma; nasopharyngeal carcinoma; gastric cancer; colon cancer; uterine cervical carcinoma; gall bladder cancer; prostate cancer; breast cancer; testicular germ cell tumors; colorectal cancer; glioma; thyroid cancer; basal cell carcinoma; gastrointestinal stromal cancer; hepatoblastoma; endometrial cancer; ovarian cancer; pancreatic cancer; renal cell cancer, Kaposi's sarcoma, chronic leukemia, sarcoma, rectal cancer, throat cancer, melanoma, colon cancer, bladder cancer, mastocytoma, mammary carcinoma, mammary adenocarcinoma, pharyngeal squamous cell carcinoma, testicular cancer, gastrointestinal cancer, or stomach cancer or urothelial cancer.

16. The method of claim 1, wherein the cancer is a drug-resistant cancer, or triple-negative breast cancer (TNBC).

17. The method of claim 1, wherein the BIK gene therapy comprises administering a BIK lipid nanoparticle with an inhibitory RNA for IRE1α.

18. The method of claim 10, wherein the inhibitory RNA for IRE1α is delivered with a viral vector, and wherein the viral vector is adenovirus vector.

19. The method of claim 10, wherein the cancer is neuroblastoma; lung cancer; bile duct cancer; non-small cell lung carcinoma; hepatocellular carcinoma; head and neck squamous cell carcinoma; squamous cell cervical carcinoma; lymphoma; nasopharyngeal carcinoma; gastric cancer; colon cancer; uterine cervical carcinoma; gall bladder cancer; prostate cancer; breast cancer; testicular germ cell tumors; colorectal cancer; glioma; thyroid cancer; basal cell carcinoma; gastrointestinal stromal cancer; hepatoblastoma; endometrial cancer; ovarian cancer; pancreatic cancer; renal cell cancer, Kaposi's sarcoma, chronic leukemia, sarcoma, rectal cancer, throat cancer, melanoma, colon cancer, bladder cancer, mastocytoma, mammary carcinoma, mammary adenocarcinoma, pharyngeal squamous cell carcinoma, testicular cancer, gastrointestinal cancer, or stomach cancer or urothelial cancer.

20. The method of claim 10, wherein the cancer is a drug-resistant cancer, or a triple-negative breast cancer (TNBC).

21. The method of claim 10, wherein the BIK gene therapy is conducted by performing administration of a BIK lipid nanoparticle in combination with an inhibitory RNA for IRE1α.
